(19) United States
(12) Patent Application Publication
(10) Pub. No.: US 2025/0022533 A1
(43) Pub. Date: Jan. 16, 2025

(54) DEGRON IDENTIFICATION USING NEURAL NETWORKS

(71) Applicant: MONTE ROSA THERAPEUTICS, INC., Boston, MA (US)

(72) Inventors: Pablo Gainza Cirauqui, Lausanne (CH); John Christopher Castle, Mainz (DE); Oliver Christof Horlacher, Olten (CH)

(21) Appl. No.: 18/709,902

(22) PCT Filed: Nov. 17, 2022
(86) PCT No.: PCT/US22/50275

§ 371 Date: May 14, 2024

Related U.S. Application Data
(60) Provisional application No. 63/280,517, filed on Nov. 17, 2021.
(60) Provisional application No. 63/419,588, filed on Oct. 26, 2022.

(51) Int. Cl. G16B 15/00; G16B 40/20

(52) U.S. Cl. CPC G16B 15/00; G16B 40/20

(57) ABSTRACT

Methods, systems, and apparatus, including computer programs encoded on a computer storage medium, for identification of degrons in various proteins.

### CLAIM OF PRIORITY

This application claims the benefit of U.S. Provisional Application Ser. No. 63/280,517, filed on Nov. 17, 2021 and U.S. Provisional Application Ser. No. 63/419,588, filed Oct. 26, 2022. The entire contents of each of the foregoing are incorporated herein by reference.

### BACKGROUND

This specification relates to processing data using machine learning models.

Machine learning models receive an input and generate an output, e.g., a prediction, based on the received input. Some machine learning models are parametric models and generate the output based on the received input and on values of the parameters of the model.

Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification describes a degron identification system and a filtering system implemented as computer programs on one or more computers in one or more locations. The identification of a degron within a protein is important for the identification of proteins that are neosubstrates for the E3 ubiquitin ligase complex, which ubiquitinates proteins and can be manipulated with small molecules to trigger targeted degradation of specific proteins of interest, including proteins that are not naturally targeted for degradation (e.g., neosubstrates). Binding of substrate proteins with the E3 ubiquitin ligase complex and subsequent destruction of the substrate is permitted if a degron present on the substrate proteins.

Throughout this specification, an “embedding” refers to an ordered collection of numerical values, e.g., a vector, matrix, or other tensor of numerical values.

A “set” of entities refers to a collection of one or more of entities.

A “degron” is a structural feature on the surface of protein that mediates recruitment of and degradation by an E3 ligase complex, e.g., an E3 ligase complex described herein. Degrons are described, for example, in Lucas and Ciulli, “Recognition of Substrate Dependent Degrons by E3 Ubiquitin Ligases and Modulation by Small-Molecule Mimicry Strategies,” Current Opinion in Structural Biology 44:101-10 (2017). For CRBN, for example, β-hairpin loop containing a glycine at a key position (G-loop) has been found as a degron based on the interaction of CK1a, GSPT1, and Zn-fingers with CRBN in their X-ray structures. See, e.g., Matyskiela et al., “A Novel Cereblon Modulator Recruits GSPT1 to the RL4 (CRBN) Ubiquitin Ligase, Nature 535 (7611):252-7 (2016); Petzold et al. “Structural basis of lenalidomide-induced CK1α degradation by the CRL4CRBN ubiquitin ligase,” Nature, 532(7597), 127-130 (2016); Furihata et al., “Structural bases of IMiD selectivity that emerges by 5-hydroxythalidomide,” Nat Commun. 11(1):4578 (2020); Sievers et al., “Defining the human C2H2 zinc finger degrome targeted by thalidomide analogs through CRBN,” Science 362(6414):eaat0572 (2018); and Wang et al., “Acute pharmacological degradation of Helios destabilizes regulatory T cells,” Nat. Chem. Bio. 17(6):711-17 (2021).

Degrons have been described and/or identified based on their primary, secondary, or tertiary protein structures. In some cases, a degron is described and/or identified in terms of their quaternary structure (e.g., in complex). In some cases, a degron is described and/or identified in the context of a crystal structure (e.g., a PDB structure). For CRBN, for example, there are six known degrons in nine crystal structures (PDB ids: 6UML, 6H0G, 6H0F, 5FQD, 5HXB, 6XK9, 7LPS, 7BQU, and 7BQV).

In some cases, the degron is a small molecule dependent degron (i.e., is a structural feature on the surface of the protein that mediates recruitment of and degradation by an E3 ligase in the presence of an E3 ligase binding modulator, e.g., an E3 ligase binding modulator described herein). In some cases, the degron is a small molecule independent degron (i.e., is a structural feature on the surface of the protein that mediates recruitment of and degradation by an E3 ligase in the absence of an E3 ligase binding modulator, e.g., an E3 ligase binding modulator described herein).

Degrons may be present on the surface of the protein target as it is expressed or added to the protein target via a linker (e.g., a proteolysis targeting chimera (PROTAC), see, e.g., Pavia and Crews, “Targeted Protein Degradation: Elements of PROTAC Design,” Curr Opin Chem Biol 50:111-19 (2019).

Degrons include, e.g., N-degrons and C-degrons, which are known and described in the art. See, e.g., Lucas and Ciulli 2017; see also, e.g., Timms and Koren, “Typing up Loose Ends: the N-degron and C-degron Pathways of Protein Degradation,” Biochem Soc Trans 48(4):1557-67 (2020).

Degrons also include, e.g., phosphodegrons and oxygen-dependent degrons (ODDs), which are also known and described in the art. See, e.g., Lucas and Ciulli 2017.

In some cases, the degron comprises or consists of the amino acid motif D-Z-G-X-Z, D-Z-G-X-X-Z, D-Z-G-X-X-X-Z, or D-Z-G-X-X-X-X-Z, wherein D is aspartic acid, each X is independently any naturally occurring amino acid, and Z is selected from the group consisting of pS (phosphorylated serine), aspartic acid, and glutamic acid.

In some cases, the degron comprises or consists of the amino acid motif X1-X2-X3-X4-X5-X6, wherein X1 is selected from the group consisting of aspartic acid, asparagine, and serine; X2 is any one of the naturally occurring amino acids; X3 is selected from the group consisting of aspartic acid, glutamic acid, and serine; X4 is selected from the group consisting of threonine, asparagine, and serine; X5 is glycine; and X6 is glutamic acid.

In some cases, the degron comprises or consists of the amino acid motif X1-X2-X3-X4-X5-X6-X7-X8-X9, wherein X1 is leucine; X2 is any one of the naturally occurring amino acids; X3 is any one of the naturally occurring amino acids; X4 is glutamine; X5 is aspartic acid; X6 is any one of the naturally occurring amino acids; X7 is aspartic acid; X8 is leucine; and X9 is glycine.

In some cases, the degron comprises or consists of the amino acid motif ETGE. In some cases, the degron comprises or consists of the amino acid motif DLG.

In some cases, the degron comprises or consists of the amino acid motif X1-X2-X3-X4-X5-X6-X7-X8, wherein X1 is phenylalanine; X2 is any one of the naturally occurring amino acids; X3 is any one of the naturally occurring amino acids; X4 is any one of the naturally occurring amino acids; X5 is tryptophan; X6 is any one of the naturally occurring amino acids; X7 is any one of the naturally occurring amino acids; and X8 is selected from the group consisting of valine, isoleucine, and leucine. In some cases the degron comprises or consisting of the amino acid motif X1-X2-X3-X4-X5-X6-X7-X8, wherein X1 is phenylalanine; X2 is any one of the naturally occurring amino acids; X3 is any one of the naturally occurring amino acids; X4 is any one of the naturally occurring amino acids; X5 is tryptophan; X6 is any one of the naturally occurring amino acids; X7 is any one of the naturally occurring amino acids; and X8 is selected from the group consisting of valine, isoleucine, and leucine forms an α-helix.

In some cases, the degron comprises or consists of the amino acid motif X1-X2-X3-X4-X5-X6, wherein X1 is leucine; X2 is any naturally occurring amino acid; X3 is any naturally occurring amino acid; X4 is leucine; X5 is alanine; and X6 is proline or hydroxylated proline (e.g., 4(R)-L-hydroxyproline).

Degrons also include, e.g., G-loop degrons. Thus, in some cases, the E3 ligase binding target is a protein comprising an E3 ligase-accessible loop, e.g., a cereblon-accessible loop, e.g., a G-loop.

In some cases, the G-loop degron comprises or consist of the amino acid sequence X1-X2-X3-X4-G-X6, wherein: each of X1, X2, X3, X4, and X6 are independently selected from any one of the natural occurring amino acids; and G (i.e. X5) is glycine.

In some cases, the G-loop degron comprises or consists of the amino acid sequence X1-X2-X3-X4-G-X6-X7, wherein: each of X1, X2, X3, X4, X6, and X7 are independently selected from any one of the natural occurring amino acids; and G (i.e. X5) is glycine.

In some cases, the G-loop degron comprises or consists of the amino acid sequence X1-X2-X3-X4-G-X6-X7-X8; wherein: each of X1, X2, X3, X4, X6, X7, and X8 are independently selected from any one of the natural occurring amino acids; and G (i.e. X5) is glycine.

In some cases, a distance from X1 to X4 is less than about 7 angstroms. In some cases, X1 and X4 are the same. In some cases, X1 is aspartic acid or asparagine and X4 is serine or threonine.

In some cases, the G-loop degron comprises or consists of the amino acid sequence X1-X2-X3-X4-G-X6, wherein X1 is selected from the group consisting of asparagine, aspartic acid, and cysteine; X2 is selected from the group consisting of isoleucine, lysine, and asparagine; X3 is selected from the group consisting of threonine, lysine, and glutamine; X4 is selected from the group consisting of asparagine, serine, and cysteine; X5 is glycine; and X6 is selected from the group consisting of glutamic acid and glutamine.

In some cases, the G-loop degron comprises or consists of the amino acid sequence X1-X2-X3-X4-G-X6, wherein X1 is asparagine; X2 is isoleucine; X3 is threonine; X4 is asparagine,; X5 is glycine; and X6 is glutamic acid.

In some cases, the G-loop degron comprises or consists of the amino acid sequence X1-X2-X3-X4-G-X6, wherein X1 is aspartic acid; X2 is lysine; X3 is lysine; X4 is serine; X5 is glycine; and X6 is glutamic acid.

In some cases, the G-loop degron comprises or consists of the amino acid sequence X1-X2-X3-X4-G-X6, wherein X1 is cysteine; X2 is asparagine; X3 is glutamine; X4 is cysteine; X5 is glycine; and X6 is glutamine.

In some cases, the degron comprises or consists of an amino acid sequence of about 2 to about 15 amino acids in length. In some cases, the degron comprises or consists of an amino acid sequence of about 6 to about 12 amino acids in length. In some cases, the degron comprises or consists of at least about 6 amino acids. In some cases, the degron comprises or consists of at least about 7 amino acids. In some cases, the degron comprises or consists of at least about 8 amino acids. In some cases, the degron comprises or consists of at least about 9 amino acids. In some cases, the amino degron comprises or consists of at least about 10 amino acids. In some cases, the G-loop degron is 6, 7, or 8 amino acids long.

The “molecular surface” of a protein refers to a solvent excluded surface of the protein.

The molecular surface of a protein can be computed using any of a variety of appropriate techniques, e.g., as described with reference to M. F. Sanner et al., “Reduced surface: an efficient way to compute molecular surfaces,” Biopolymers 38, 305-320 (1996).

A continuous protein molecular surface can be discretely represented as a polygon mesh defined by: (i) a set of vertices, (ii) a set of edges, and (iii) a set of polygonal (e.g., triangular) faces. Each vertex in the mesh represents a respective point on the protein molecular surface and can be defined, e.g., by three-dimensional (3-D) Cartesian (x-y-z) spatial coordinates. Each edge in the mesh connects a respective pair of vertices in the mesh. Each polygonal face in the mesh represents a two-dimensional (2-D) region (e.g., triangular region) enclosed by a respective closed set of edges in the mesh.

A “geodesic distance” between two points (vertices) on a protein molecular surface refers to the length of the shortest path (curve) along the protein molecular surface that connects the two points. On a discrete representation of a protein molecular surface as a mesh, the geodesic distance between two vertices can be measured as the shortest sequence of adjacent mesh edges connecting the two vertices. The length of an edge can refer to the Euclidean distance between the vertices connected by the edge. The geodesic distance between vertices on a protein molecular surface can be computed, e.g., using the Dijkstra algorithm.

Each vertex of a protein molecular surface can be associated with a respective set of features. The features for a vertex can include, e.g., chemical features (e.g., that characterize biochemical properties of the protein molecular surface at the vertex), geometric features (e.g., that characterize the shape of the protein molecular surface at the vertex), or both.

Chemical features for a vertex can be learned directly from the data or can be precomputed include, e.g., one or more of: a hydropathy index feature, a continuum electrostatics feature, or a hydrogen bonding potential feature. A hydropathy index for a vertex can represent a degree to which the amino acid in the protein closest to the vertex is hydrophobic or hydrophilic (e.g., according to the Kyte-Doolittle scale). A continuum electrostatics feature for a vertex can represent an electrostatic charge value at the vertex (e.g., computed using the Poisson-Boltzmann equation). A hydrogen bonding potential feature can represent a potential for formation of a hydrogen bond near the vertex (e.g., based on an orientation between heavy atoms near the vertex, e.g., as described with T. Kortemme et al., “An orientation-dependent hydrogen bonding potential improves prediction of specificity and structure for proteins and protein-protein complexes,” J. Mol. Biol. 326, 1239-1259 (2003)).

Geometric features for a vertex can be learned from the data or precomputed to include, e.g., a shape index feature or distance-dependent curvature features. A shape index feature for a vertex can measure the concavity or convexity of the protein molecular surface at the vertex and can be computed, e.g., as:

$$\frac{2}{π} {tan}^{- 1} \frac{{κ}_{1} + {κ}_{2}}{{κ}_{1} - {κ}_{2}} \qquad ( 1 )$$

where κ₁ and κ₂ are the principal curvatures of the protein molecular surface at the vertex. Distance-dependent curvature features for a vertex can represent a distribution of curvatures of the protein molecular surface within a predefined geodesic distance of the vertex, e.g., as described with reference to S. Yin et al., “Fast screening of protein surfaces using geometric invariant fingerprints,” Proceedings of the National Academy of Sciences, September 2009, 106 (39) 16622-16626.

A surface patch on a protein molecular surface refers to a region of the protein molecular surface. For example, a surface patch on a protein molecular surface can refer to a region of the protein molecular surface within a predefined geodesic distance (e.g., 9 Angstroms or 12 Angstroms) of a center point of the surface patch. For convenience, throughout this specification, a patch on a protein molecular surface may be referred to as a “surface patch” or simply a “patch.”

A surface patch on a protein molecular surface can be parameterized using any appropriate local frame of reference for the surface patch. That is, the position of any point within the surface patch can be represented by a respective set of coordinates in the local frame of reference of the surface patch. For example, a surface patch can be parametrized by geodesic polar coordinates. In geodesic polar coordinates, the position of any point within the surface patch can be represented by coordinates (r, θ), where r represents the geodesic distance of the point from the center of the surface patch, and θ represents an angular position of the point relative to a predefined axis (direction) from the center of the surface patch.

Generally, angular position in a geodesic polar coordinate system can be measured with respect to any predefined axis from the center of the surface patch. A geodesic polar coordinate system for a surface patch can be “rotated,” i.e., by rotating the orientation of axis used to measure the angular position of points in the geodesic polar coordinate system.

Angular position can be computed, for example, as described in Gainza et al., “Deciphering Interaction Fingerprints From Protein Molecular Surfaces Using Geometric Deep Learning,” Nature Methods 17:184-92 (2020), as follows: A classical multidimensional scaling algorithm (O'Connell, A. A., Borg, I. & Groenen, P. Modern multidimensional scaling: theory and applications J. Am. Stat. Assoc. 94, 338-339 (2006)) was used to flatten patches into the plane based on the Dijkstra approximation to pairwise geodesic distances between all vertices. As molecular surface patches have no canonical orientation, a random direction in the computed plane was chosen as a reference and the angle of each vertex to this reference in the plane was set as the angular coordinate.

In some cases, angular position is computed using a much faster algorithm described as follows. The angles between the center vertex and each immediately connected neighbor are computed with respect to an arbitrary chosen direction. Then the rest of the patch is explored using a bread-first search algorithm (Even, Shimon. Graph algorithms. Cambridge University Press, 2011), that is prepopulated with the center node and its immediate neighbors. As the algorithm expands nodes in the breadth-first search algorithm, the angle at each node that is expanded is the average of every angle assigned to each previously visited node in the bread-first search exploration.

Data defining a surface patch on a protein molecular surface can include, for each vertex included in the surface patch: (i) the coordinates of the vertex in the frame of reference of the surface patch, and (ii) the features of the vertex. The coordinates of the vertex in the frame of reference of the surface patch can be, e.g., geodesic polar coordinates, and the features of the vertex can include, e.g., chemical and geometric features, as described above.

The degron identification system described in this specification can identify one or more protein molecular surface patches, from a set “candidate” surface patches, as being “predicted degron” surface patches, i.e., that are each predicted to correspond to a degron on a respective protein molecular surface.

The filtering system can apply a filtering criterion to the set of predicted degron surface patches to identify and remove “false positives” from the set of predicted degron surface patches, i.e., candidate surface patches that are incorrectly predicted to be degron surface patches. In particular, the filtering system can identify a predicted degron surface patch as a false positive if the predicted degron surface patch does not include structural features (e.g., G-loops) that are characteristic of degrons. When a predicted degron is identified in a protein, the protein is identified as a potential neosubstrate which then can be used in the design of novel therapeutic agents for treating cancer or other diseases. The novel therapeutic agents can bind to an E3 ligase which will then target the neosubsrate for degradation.

Particular embodiments of the subject matter described in this specification can be implemented so as to realize one or more of the following advantages.

The degron identification system described in this specification can predict whether a candidate surface patch from a protein molecular surface corresponds to a degron by comparing the candidate surface patch to a degron surface patch (i.e., that is known to correspond to a degron) in an embedding space. In particular, the degron identification system computes respective embeddings of both surface patches using an embedding neural network. The degron identification system then predicts whether the candidate surface patch corresponds to a degron based on a similarity measure between the respective embeddings of the candidate surface patch and the degron surface patch in the embedding space.

The degron identification system described in this specification can also predict whether a candidate surface patch from a protein molecular surface corresponds to a degron by comparing the candidate surface patch to an E3 ligase surface patch (i.e., that is a surface or neosurface of an E3 ligase substrate receptor protein) in an embedding space. In particular, the degron identification system computes respective embeddings of both surface patches using an embedding neural network. The degron identification system then predicts whether the candidate surface patch corresponds to a degron based on a complementarity measure between the respective embeddings of the candidate surface patch and the E3 ligase surface patch in the embedding space.

Generally, direct comparison of protein surface patches can be difficult, e.g., because each protein surface patch can include a variable number of vertices located at variable locations within the protein surface patch. By computing fixed-dimensionality embeddings of protein surface patches and then comparing protein surface patches in the embedding space, the degron identification system facilitates rapid and efficient comparison of protein surface patches, irrespective of the number and location of the vertices in the protein surface patches.

The filtering system described in this specification can increase the accuracy and quality of a set of predicted degron surface patches generated by the degron identification system by removing surface patches that do not include a specified structural feature, e.g., that is known to be characteristic of degrons. The filtering system thus enables more efficient use of resources, e.g., computational resources such as memory and computing power, by preventing downstream processing of surface patches that are incorrectly identified as being degron surface patches.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example degron identification system and an example filtering system.

FIG. 2A shows an example degron identification system.

FIG. 2B shows an example degron identification system.

FIG. 2C shows an example degron identification system.

FIG. 3 illustrates an example architecture of an embedding neural network that is configured to process data defining a surface patch on a protein molecular surface to generate an embedding of the surface patch.

FIG. 4 shows an example embedding training system.

FIG. 5 shows an example filtering system.

FIG. 6A is a flow diagram of an example process for classifying one or more candidate protein molecular surface patches as corresponding to degrons.

FIG. 6B is a flow diagram of an example process for classifying one or more candidate protein molecular surface patches as corresponding to degrons.

FIG. 6C is a flow diagram of an example process for classifying one or more candidate protein molecular surface patches as corresponding to degrons.

FIG. 7 is an example of the architecture of a structural classification neural network.

FIG. 8 shows an example of training a degron identification machine learning agent using surface patches.

FIG. 9 shows a conceptual example of using an ultra-fast fingerprint search for similar surfaces, finding surface that mimic known degron surfaces.

FIG. 10 depicts a E3:MGD neosurface for an ultra-fast fingerprint search for complementary surfaces, such as for E3 ligase-neosubstrate matchmaking.

FIG. 11 depicts an example of a method for learning CRBN degron features from known degron surfaces. The algorithm classifies protein surfaces for the presence of degrons. The algorithm creates a feature-rich surface characterization and uses 3 layers of geodesic convolution with deep vertexes to classify input surfaces.

FIG. 12 depicts an example of a yeast-3-hybrid proximity assay. The assay identifies MGD-induced interactions between CRBN and cDNA library-derived targets. It maps degrons to individual domains.

FIG. 13 shows that 8 novel G-loops from 5 distinct domain classes, identified using yeast 3 hybrid experiments, match predictions made by a method for learning CRBN degron features from known degron surfaces.

FIG. 14 shows that a degron surface found and characterized using methods described herein has a unique G-loop surface; the spider plots show summary statistics for each of the input features of the neural network.

FIG. 15 shows that the unique G-loop identified in FIG. 14 enables selective MGD degradation.

FIG. 16 shows an example of encoding protein surfaces as fingerprints, which enables ultra-fast, proteome-wide searching for similar & complementary fingerprints of degrons and E3 ligases respectively.

FIG. 17 shows an example of a full pipeline to search for complementary or similar degrons.

FIG. 18 shows that fingerprint encoding enables ultra-fast searching of, for example, proteome-wide queries of either complementary or similar surfaces of degrons and E3 ligases respectively.

FIG. 19 shows an example of proteome-wide fast matching of degron surface mimics by matching of surface fingerprints (and not, e.g., G-loops per se).

FIG. 20 shows an example of a novel degron identified by a mimicry search, with shading for similarity to an established G-loop degron. The degron is a non-hairpin, non-canonical degron in an established oncology target.

FIG. 21 shows that NanoBRET confirmed the prediction and binding mode.

FIG. 22 is an example of how the E3 ligase neosurface footprint can be used to find novel neosubstrates (as it defines the target-complementary surface).

FIG. 23 shows an example of a method for finding proteins complementary to E3 ligases. In this example, the E3 ligase footprint is encoded as a fingerprint for fast E3-target matchmaking.

FIG. 24 shows an example of how the methods described herein expand the target space to non-canonical degrons.

Like reference numbers and designations in the various drawings indicate like elements.

### DETAILED DESCRIPTION

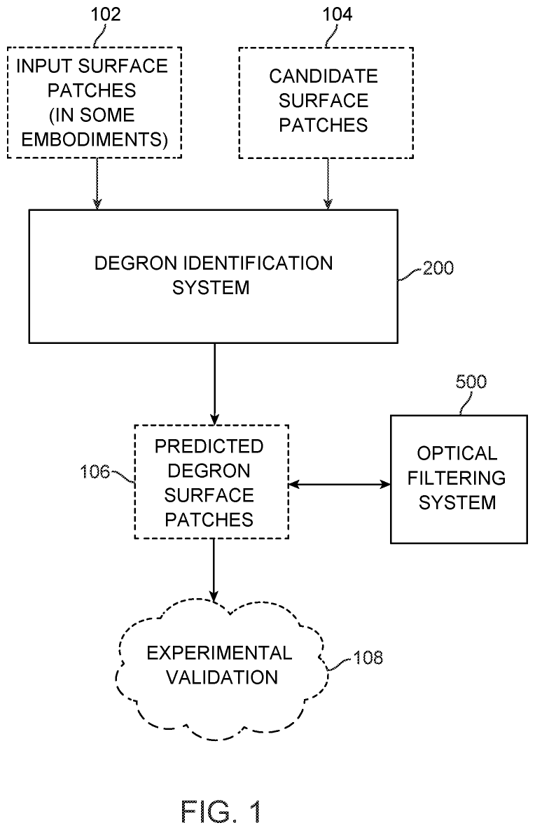

FIG. 1 shows an example degron identification system 200 and an example filtering system 500.

The degron identification system 200 (e.g., the degron identification system 200A, 200B, or 200C) and the filtering system 500 are used to identify a set of predicted degron surface patches 106, i.e., that are each predicted to correspond to a degron on a respective protein molecular surface.

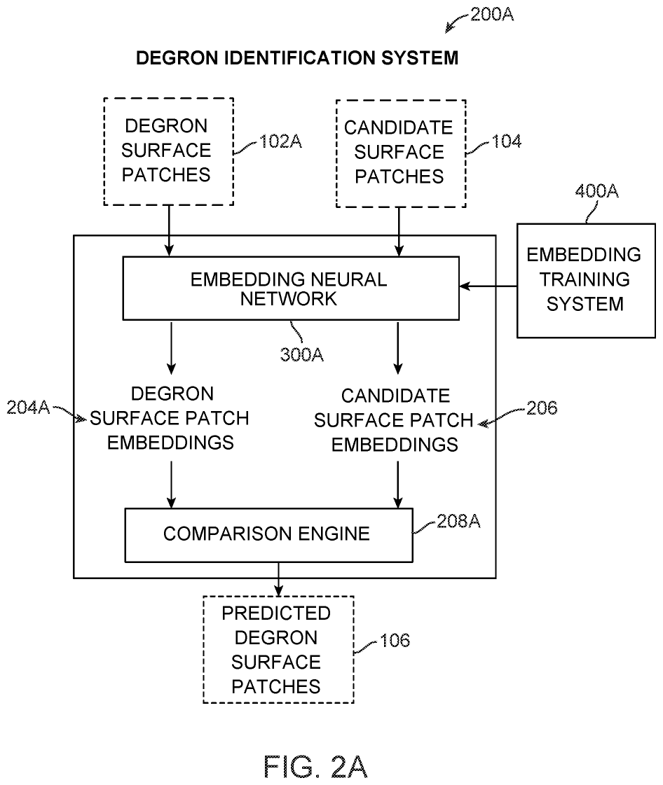

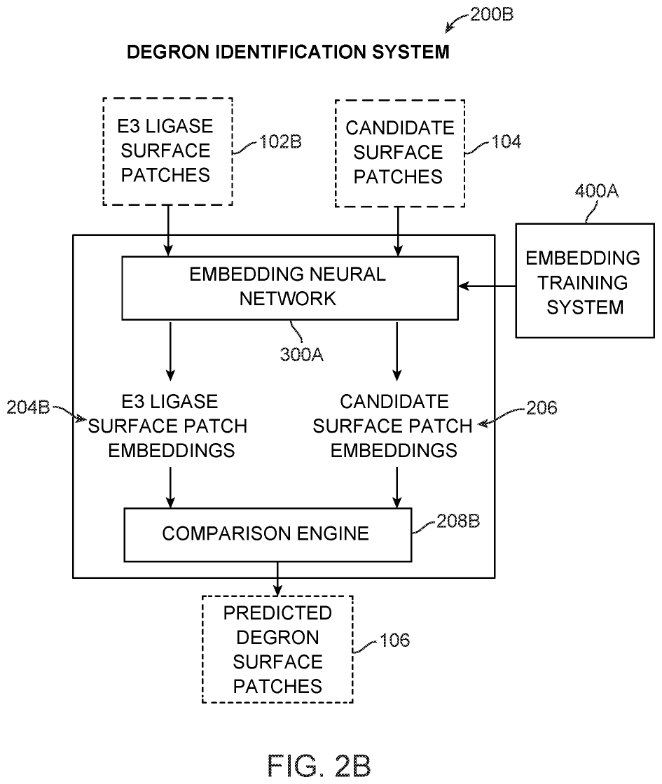

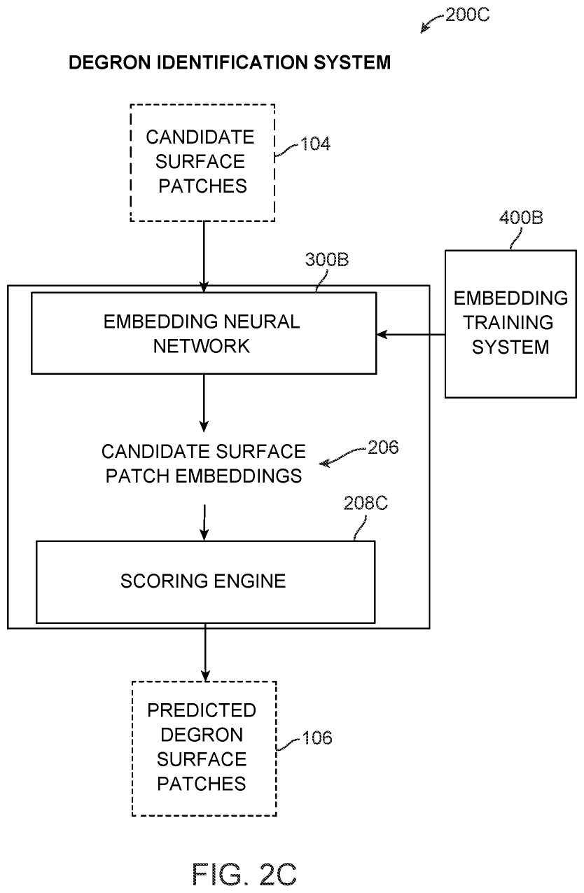

The degron identification system 200, which is described in more detail with reference to FIG. 2A, FIG. 2B, and 2C, generates a respective embedding of each surface patch in a set of “candidate surface patches” 104, and in some embodiments, a set of “input patches” 102 (e.g., 102A, 102B). The input patches 102 and the candidate surface patches 104 are surface patches on respective protein molecular surfaces. In some cases, the input surface patch 102 is a degron surface patch 102A, known to correspond to a degron on a protein molecular surface. In some cases, the input surface patch 102 is a E3 ligase surface patch 102B, e.g., corresponding to an E3 ligase substrate receptor protein molecular surface or neosurface.

The degron identification system 200A computes a similarity measure between the embeddings of the degron surface patches and the embeddings of the candidate surface patches, and can classify one or more of the candidate surface patches as being predicted degron surface patches 106 based on the similarity measure.

The degron identification system 200B computes a complementarity measure between the embeddings of the E3 ligase surface patches and the embeddings of the candidate surface patches, and can classify one or more of the candidate surface patches as being predicted degron surface patches 106 based on the complementarity measure.

The degron identification system 200C is trained to learn from the surface of proteins that correspond to degrons (known and/or predicted), and can classify one or more of the candidate surface patches 106.

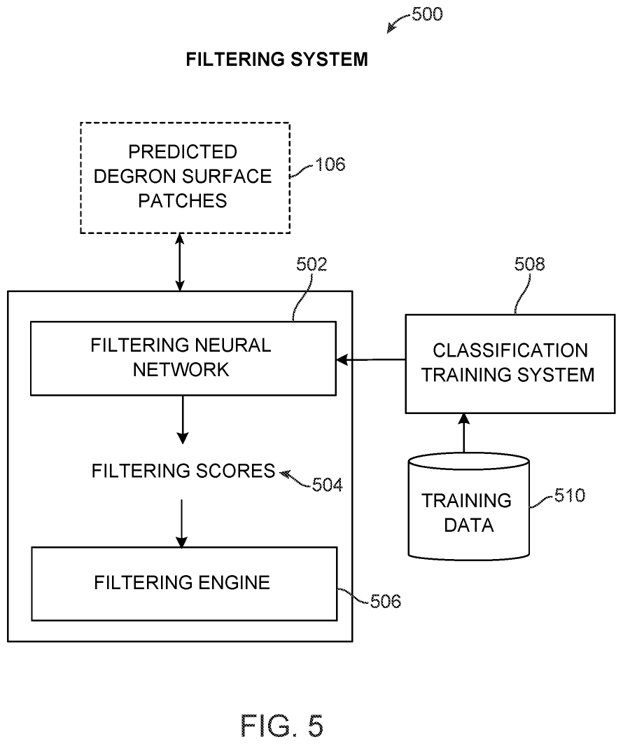

The filtering system 500, which is described in more detail with reference to FIG. 5, generates a respective “filtering score” for each predicted degron surface patch 106. A filtering score for a predicted degron surface patch represents a likelihood that the predicted degron surface patch 106 includes a structural feature known to be characteristic of degrons, e.g., a G-loop. The filtering system in 500 can be a system that implements a neural network (NN) trained on sets of G-loops and non-G-loop regions of proteins, and trained to classify G-loops purely based on their surface. The purpose of the network is to further select only surface-based degrons that have high surface mimicry to a G-loop. The filtering system is then applied to all the degrons identified based on their surface. Only those surface degrons that achieve a sufficiently high score (e.g., >0.9 or another specified threshold) by the filtering system 500 are accepted as surface degrons, while those below this score can be discarded.

FIGS. 2A, 2B, and 2C show examples (200A and 200B, and 200C, respectively) of the degron identification system 200. The degron identification system 200 is an example of a system implemented as computer programs on one or more computers in one or more locations in which the systems, components, and techniques described below are implemented.

The degron identification system 200 is configured to receive data defining: a set of candidate surface patches 104, and, in some cases, a set of input surface patches 102 (e.g., degron surface patches 102A or E3 ligase surface patches 102B).

Each degron surface patch 102A is a surface patch, on a respective protein molecular surface, that is known to correspond to a degron and/or predicted to correspond to a degron.

Each E3 ligase surface patch 102B is a surface patch, on an E3 ligase substrate receptor protein, such as CRBN, that corresponds to a surface (unbound) or neosurface (e.g., molecuclar glue bound) of the E3 ligase substrate receptor protein.

Each candidate surface patch 104 is a surface patch on a respective protein molecular surface. The candidate surface patches 104 can be obtained from the respective molecular surfaces of one or more proteins. For example, the set of candidate surface patches 104 can include surface patches that are systematically extracted from the molecular surfaces of one or more proteins.

Systematically extracting surface patches from a protein molecular surface can include, e.g., extracting surface patches that cover the protein molecular surface, e.g., such that every vertex in a discrete representation of the protein molecular surface as a polygon mesh is included in at least one surface patch. For example, surface patches can be systematically extracted from a protein molecular surface by extracting a respective surface patch centered on each vertex in a discrete representation of the protein molecular surface as a polygon mesh. A surface patch centered on a vertex can be, e.g., a region of the protein molecular surface within a predefined geodesic distance of the vertex on the protein molecular surface.

The degron identification system 200 processes the set of candidate surface patches 104, and, in some cases, the set of input surface patches 102, to identify one or more of the candidate surface patches 104 as being predicted degron patches 106. A predicted degron patch 106 is a surface patch on a protein molecular surface that is predicted to correspond to a degron.

The degron identification system 200A can identify a candidate surface patch 104 as being a predicted degron surface patch 106 based on a measure of similarity, measured in an embedding space, between the candidate surface patch 104 and a degron surface patch 102A, as will be described in more detail below.

The degron identification system 200B can identify a candidate surface patch 104 as being a predicted degron surface patch 106 based on a measure of complementarity, measured in an embedding space, between the candidate surface patch 104 and an E3 ligase surface patch 102B, as will be described in more detail below.

The degron identification system 200C can identify a candidate surface patch 104 as being a predicted degron surface patch 106, as will be described in more detail below.

The degron identification system 200 includes an embedding neural network 300 and a comparison engine 208, which are each described in more detail next.

The embedding neural network 300 is configured to process data defining a surface patch from a protein molecular surface, in accordance with values of a set of embedding neural network parameters, to generate an embedding of the surface patch in an embedding space (i.e., a space of possible embeddings). The embedding space can be any appropriate space, e.g., a 10 dimensional, 50 dimensional, or 100 dimensional Euclidean space.

An embedding of a surface patch encodes the features associated with the vertices included in the surface patch in a fixed-dimensional representation (e.g., that is independent of the number of vertices included in the surface patch).

In some cases, the embedding neural network 300 is trained, by an embedding training system 400 (e.g., 400A or 400B).

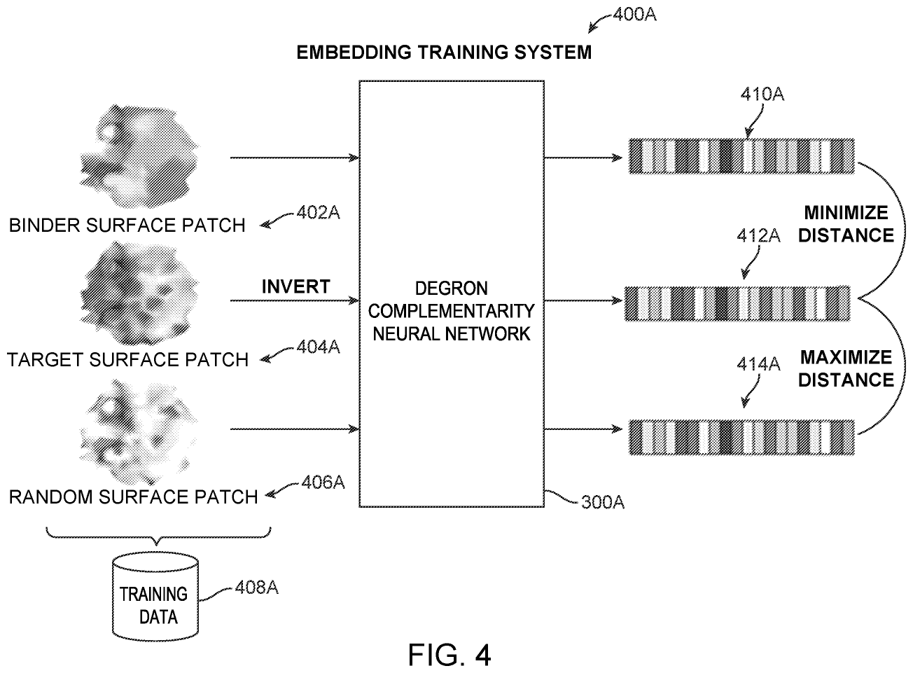

In some cases, the embedding neural network 300 is a degron complementarity neural network (300A), trained by an embedding training system 400A, to generate “similar” embeddings (e.g., according to an appropriate similarity measure in the embedding space) of “similar” surface patches (e.g., that share similar geometric and chemical features). Conversely, the embedding training system 400A trains the degron complementarity neural network 300A to generate dissimilar embeddings of dissimilar surface patches. An example of an embedding training system 400A is described in more detail with reference to FIG. 4A.

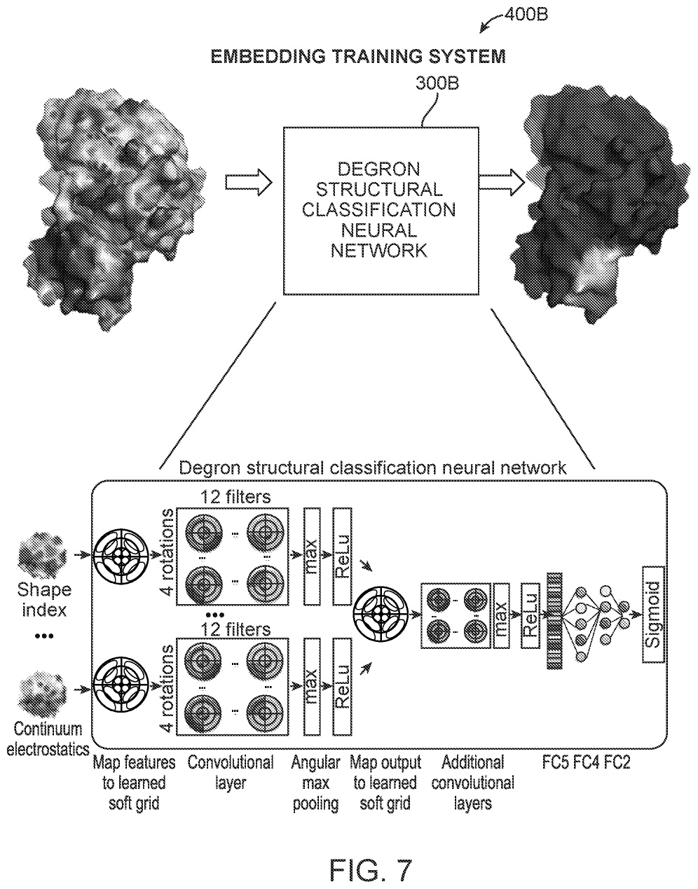

In some cases, the embedding neural network 300 is a degron structural classification neural network (300B), trained by an embedding training system 400B, to generate on sets of degron (e.g., G-loop) and non-degron regions of proteins, to classify degrons purely based on their surface. The degron structural classification neural network 300B may have an architecture such as that depicted in FIG. 7, where during training the network receives as input a protein's surface such that a small fraction of the surface contains a degron (e.g., G-loop). The neural network processes the surface and is trained to predict a particular region of the surface that contains the degron (e.g., G-loop). In some cases, the input protein surfaces comprise known degron surface patches, In some cases, the input protein surfaces comprise augmented degron surface patches. In some cases, the input protein surfaces comprise both known and augmented degron surface patches. Augmented degron surface patches are predicted degron surface patches that, in some cases, are generated according to method 600A, e.g., as carried out by degron identifications system 200A.

The input during training of the degron structural classification neural network 300B is a protein surface, with per-vertex features (shape index, distance dependent curvature, APBS electrostatics, hydrophobicity, and free/proton electrons), as well as a system of geodesic polar coordinates (angular and radial) for each decomposed patch from the surface. In the forward pass, the surface passed over three layers of geodesic convolution, and the output layer was a sigmoid activation function (details of the architecture are shown in FIGS. 7, 8, and 9). As a loss function, a binary cross entropy loss function was used to minimize the difference between the ground truth degron of the training neosubstrate, and the predicted degron surface. In the backward pass, the weights of the neural network were optimized using an Adam optimizer.

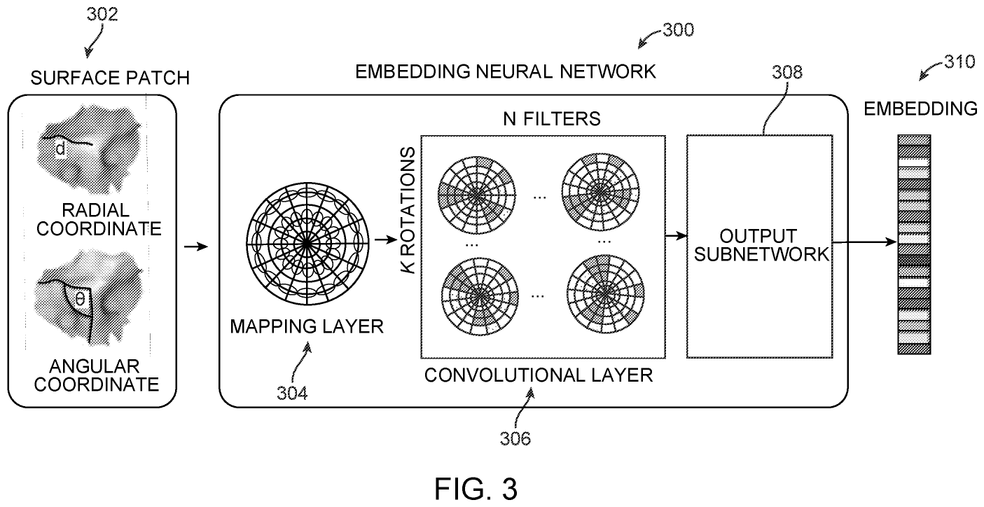

The embedding neural network 300 can have any appropriate neural network architecture that enables it to perform its described functions, e.g., processing surface patches to generate embeddings. In particular, the embedding neural network can include any appropriate types of neural network layers in any appropriate numbers and connected in any appropriate configuration. An example architecture of an embedding neural network is described in more detail with reference to FIG. 3.

The degron identification system 200 uses the embedding neural network 300 to generate embeddings of the candidate surface patches 104 and, in some cases, the input surface patches 102.

In some cases, the embedding neural network 300A processes respective neural network inputs representing each degron surface patch 102A to generate a corresponding degron surface patch embedding 204A, and the embedding neural network 300A processes each candidate respective neural network inputs representing each surface patch 104 to generate a corresponding candidate surface patch embedding 206. In general, embedding neural network 300A processes inputs representing each degron surface patch 102A and each candidate surface patch 104 individually. Thus, in some applications, the degron identification system 200A need only process the set of degron surface patches 102A once to generate a set of degron surface patch embeddings 204A that can be used as a reference for comparison against embeddings 206 for different sets of candidate surface patches 104 generated at a different times. By re-using all or some of the degron surface patch embeddings 204A for comparison against different candidate surface patch embeddings 206, the degron identification system 200A can more efficiently identify predicted degron surface patches 106 without re-processing inputs representing the same degron surface patches 102A each time. For example, the degron identification system 200A can store degron surface patch embeddings 204A for future use in an internal memory of the system 200A or in an external memory device that interfaces with the degron identification system 200A.

In some cases, the embedding neural network 300A processes respective neural network inputs representing each E3 ligase surface patch 102B to generate a corresponding E3 ligase surface patch embedding 204B, and the embedding neural network 300A processes each candidate respective neural network inputs representing each surface patch 104 to generate a corresponding candidate surface patch embedding 206. In general, embedding neural network 300A processes inputs representing each E3 ligase surface patch 102B and each candidate surface patch 104 individually. Thus, in some applications, the degron identification system 200B need only process the set of E3 ligase surface patches 102B once to generate a set of E3 ligase surface patch embeddings 204B that can be used as a reference for comparison against embeddings 206 for different sets of candidate surface patches 104 generated at a different times. By re-using all or some of the E3 ligase surface patch embeddings 204B for comparison against different candidate surface patch embeddings 206, the degron identification system 200B can more efficiently identify predicted degron surface patches 106 without re-processing inputs representing the same E3 ligase surface patches 102B each time. For example, the degron identification system 200B can store E3 ligase surface patch embeddings 204B for future use in an internal memory of the system 200B or in an external memory device that interfaces with the degron identification system 200B.

In some cases, the embedding neural network 300B processes each candidate respective neural network inputs representing each surface patch 104 to generate a corresponding candidate surface patch embedding 206.

The comparison engine 208A is configured to compute a respective similarity measure between: (i) each degron surface patch embedding 204A, and (ii) each candidate surface patch embedding 206. The similarity measure can be, e.g., a Euclidean similarity measure, a cosine similarity measure, or any other appropriate similarity measure. In some implementations, comparison engine 208A can apply selection criteria to select only a subset (e.g., less than all) of the degron surface patch embeddings 204A for comparison against one or more candidate surface patch embeddings 206. In some implementations, comparison engine 208A can apply selection criteria to select only a subset (e.g., less than all) of the candidate surface patch embeddings 206A for comparison against one or more degron surface patch embeddings 204A.

The comparison engine 208A can then identify one or more of the candidate surface patches 104 as being predicted degron surface patches 106 based on the similarity measures between the degron surface patch embeddings 204A and the candidate surface patch embeddings 206. For example, the comparison engine 208A can identify a candidate surface patch 104 as being a predicted degron surface patch 106 if the similarity measure between the embedding of the candidate surface patch 104 and the embedding of at least one degron surface patch 102A satisfies (e.g., exceeds) a threshold.

The comparison engine 208B is configured to compute a respective complementarity measure between: (i) each degron surface patch embedding 204B, and (ii) each candidate surface patch embedding 206. The complementarity measure can be, e.g., a Euclidean similarity measure, a cosine similarity measure, or any other appropriate similarity measure. In some implementations, the comparison metric consists of an alignment step with, for example the RANSAC algorithm followed by a neural network that receives as input the embeddings for the two patches and scores the alignment. In some cases, comparison engine 208B can apply selection criteria to select only a subset (e.g., less than all) of the degron surface patch embeddings 204B for comparison against one or more candidate surface patch embeddings 206. In some implementations, comparison engine 208B can apply selection criteria to select only a subset (e.g., less than all) of the candidate surface patch embeddings 206B for comparison against one or more degron surface patch embeddings 204B.

The comparison engine 208B can then identify one or more of the candidate surface patches 104 as being predicted degron surface patches 106 based on the complementarity measures between the E3 ligase surface patch embeddings 204B and the candidate surface patch embeddings 206. For example, the comparison engine 208B can identify a candidate surface patch 104 as being a predicted degron surface patch 106 if the complementarity measure between the embedding of the candidate surface patch 104 and the embedding of at least one E3 ligase surface patch 102B satisfies (e.g., exceeds) a threshold.

The scoring engine 208C is configured to compute a prediction score for each candidate surface patch embedding 206. The scoring engine 208C can then identify one or more of the candidate surface patches 104 as being predicted surface patches 106 based on the prediction score. For example, the scoring engine 208C can identify a candidate surface patch 104 as being a predicted degron surface patch 106 if the prediction score satisfies (e.g., exceeds) a threshold.

The value of the threshold can be set in any appropriate way. For example, setting the threshold to a higher value may establish a more stringent criterion for identifying a candidate surface patch 104 as being a predicted degron surface patch 106, thus reducing the likelihood of false positives among the predicted degron surface patches 106. Similarly, setting the threshold to a lower value may establish a less stringent criterion for identifying a candidate surface patch 104 as being a predicted degron surface patch 106, thus reducing the likelihood of excluding true samples among the predicted degron surface patches 106.

After identifying the predicted degron surface patches 106, the degron identification system 200 can provide data identifying the predicted degron surface patches 106, e.g., to a user of the degron identification system 200. In some cases, the degron identification system 200 may identify none of the candidate surface patches 104 as being predicted degron surface patches 106, which can also be indicated, e.g., to a user of the degron identification system 200.

Optionally, the set of predicted degron surface patches 106 can be provided to a filtering system, e.g., the filtering system 500 described with reference to FIG. 5, to identify and remove false positives from the set of predicted degron surface patches 106.

FIG. 3 illustrates an example architecture of an embedding neural network 300 that is configured to process data defining a surface patch 302 on a protein molecular surface to generate an embedding 310 of the surface patch 302. For example, surface patch 302 can be a degron surface patch 102 or a candidate surface patch 104. The embedding 310 of the surface patch 310 can be a degron surface patch embedding 204 or a candidate surface patch embedding 206.

The surface patch 302 can be parametrized using geodesic polar coordinates, i.e., such that the position of any point within the surface patch 302 can be represented by: (i) a radial coordinate r, and (ii) an angular coordinate θ. The radial coordinate of a point represents the geodesic distance of the point from the center of the surface patch. The angular coordinate of a point represents an angular position of the point relative to a predefined axis (direction) from the center of the surface patch.

In particular, each vertex in the surface patch can be associated with respective angular and radial coordinates representing the position of the vertex in the patch. Each vertex is also associated with a set of features, e.g., chemical and geometric features, characterizing the protein molecular surface at the vertex. In some cases, the geometric feature(s) are selected from the group consisting of a shape index (Koenderink et al., “Surface Shape and Curvature Scales,” Image Vis. Comput. 10:557-64 (1992), which is hereby incorporated by reference in its entirety), distance-dependent curvature (Yin et al., “Fast Screening of Protein Surfaces using Geometric Invariant Fingerprints” Proc. Natl. Acad. Sci. USA 106:16622-26 (2009), which is hereby incorporated by reference in its entirety), geodesic polar coordinate(s), and combinations thereof.

In some cases, the chemical feature(s) are selected from the group consisting of hydropathy index (Kyte et al., “A Simple Method for Displaying the Hydropathic Character of a Protein” J. Mol. Biol. 157:105-32 (1982)), continuum electrostatics (Jurrus et al. “Improvements to the APBS Biomolecular Solvation Software Suite,” Protein Sci. 27:112-28 (2018), which is hereby incorporated by reference in its entirety), location of free electrons (Kortemme et al., “An Orientation-Dependent Hydrogen Bonding Potential Improves Prediction of Specificity and Structure for Proteins and Protein-Protein Complexes,” J. Mol. Biol. 326:1239-59 (2003), which is hereby incorporated by reference in its entirety), location of free proton donors (Kortemme et al., “An Orientation-Dependent Hydrogen Bonding Potential Improves Prediction of Specificity and Structure for Proteins and Protein-Protein Complexes,” J. Mol. Biol. 326:1239-59 (2003), which is hereby incorporated by reference in its entirety), and combinations thereof.

The embedding neural network includes a mapping layer 304, a convolutional layer 306, and an output subnetwork 308.

The mapping layer 304 is configured to map the input surface patch 302 to a fixed-size representation, referred to as a “polar grid” representation, that includes an array of “grid cells,” where each grid cell can be represented as a vector of numerical values. (It can be appreciated that the surface patch 302 can include a variable number of vertices at variable positions in the surface patch 302 and therefore its representation, prior to being processed by the mapping layer 304, is not fixed-size.)

Each grid cell in the polar grid representation of the surface patch 302 corresponds to a respective weight function, i.e., that associates each pair of radial and angular coordinates (r, θ) in the surface patch with a respective weight value. For example, the weight function can be a respective (2-D) Gaussian kernel parameterized by a mean vector and a covariance matrix. To generate each grid cell, the mapping layer 304 combines the features associated with the vertices in the surface patch 302 using the corresponding weight function, e.g., as:

$$C = {\sum }_{y} w ( {( r , θ )}_{y} ) \cdot f ( y ) \qquad ( 2 )$$

where C is a grid cell, y indexes the vertices in the surface patch, w(·) is the weight function corresponding to the grid cell, (r, θ)_{y} are the radial and angular coordinates of vertex y, and f(y) are the features associated with vertex y.

Generally, a polar grid representation of the surface patch 302 depends on the arbitrary orientation of the geodesic polar coordinate system used to parameterize the positions of the vertices in the surface patch 302. (The orientation of the geodesic polar coordinate system refers to the orientation of the axis relative to which the angular position of each vertex in the surface patch is measured in the geodesic polar coordinate system). Therefore, optionally, the embedding neural network 300 can use the mapping layer 304 to generate multiple (K) polar grid representations of the surface patch, where each polar grid representation is generated with respect to a geodesic polar coordinate system rotated to a different angle. Processing the multiple polar grid representations of the surface patch 302 using the subsequent layers of the embedding neural network 300 can achieve (approximate) invariance of the output embedding 310 to the arbitrary orientation of the geodesic polar coordinate system used to parametrize the surface patch 302.

The convolutional layer 306 includes N “filters.” Each filter is configured to process a polar grid representation of the surface patch 302, e.g., by computing a linear combination of the grid cells in the polar grid representation, where the values of the coefficients multiplying the grid cells in the linear combination are parameters of the filter.

The convolutional layer 306 can apply each filter to each polar grid representation of the surface patch 302, and then combine the outputs of the filters using a pooling operation, e.g., a max-pooling operation. Optionally, the convolutional layer can apply a non-linear activation function, e.g., a rectified linear unit (ReLU) activation function, to each component of the output of the pooling operation.

The output subnetwork 308 is configured to process the output of the convolutional layer 306 to generate the embedding 310. The output subnetwork 308 can include, e.g., one or more fully-connected neural network layers, or any other appropriate neural network layers.

The learnable parameters of the embedding neural network 300 can include, e.g., the parameters of the weight functions of the mapping layer 304, the parameters of the filters of the convolutional layer 306, and the parameters of the output subnetwork 308. The learnable parameters of the embedding neural network 300 can be trained by an embedding training system, as will be described in more detail next with reference to FIG. 4 and FIG. 7.

FIG. 4 shows an example embedding training system 400A. FIG. 7 shows an example embedding training system 400B. The embedding training systems 400A and 400B are examples of a system implemented as computer programs on one or more computers in one or more locations in which the systems, components, and techniques described below are implemented.

The embedding training system 400 is configured to train the embedding neural network 300, i.e., to determine trained values of the embedding neural network parameters from initial values of the embedding neural network parameters. Thus, the embedding training system 400A is configured to train the degron complementarity neural network 300A, i.e., to determine trained values of the embedding neural network parameters from initial values of the embedding neural network parameters. Likewise, the embedding training system 400B is configured to train the degron structural classification neural network 300B.

The embedding training system 400A trains the embedding neural network 300 (in this case, the degron complementarity neural network 300A) on a set of training data 408A that includes multiple training examples. Each training example includes a respective pair of protein surface patches, i.e., that are each surface patches from respective protein molecular surfaces. Certain training examples, referred to for convenience as “positive” training examples, include a “binder” surface patch and a “target” surface patch that are known (e.g., from experimental techniques) or predicted (e.g., from in silico modeling) to be interacting surface patches, e.g., that bind to one another. Other training examples, referred to for convenience as “negative” training examples, include a “target” surface patch and a “random” surface patch, where the random surface patch is a randomly selected surface patch that is understood not to interact (e.g., bind) with the target surface patch.

The embedding training system 400B trains the embedding neural network 300 (in this case, the degron structural classification neural network 300B) on a set of training data. The degron structural classification neural network 300B may have an architecture such as that depicted in FIG. 7, where during training the network receives as input a protein's surface such that a small fraction of the surface contains a degron (e.g., G-loop). The neural network processes the surface and is trained to predict a particular region of the surface that contains the degron (e.g, G-loop).

Prior to training the embedding neural network 300, the embedding training system 400 can initialize the values of the embedding neural network parameters, e.g., by setting each parameter to a random value.

After initializing the parameter values of the embedding neural network 300, the embedding training system 400 can train the embedding neural network over a sequence of training iterations. At each training iteration, the embedding training system 400 can sample a “batch” (set) of training examples from the training data 408, and train the embedding neural network 300 on each training example in the batch. Each batch of training examples includes a set of positive training examples and a set of negative training examples.

To train the embedding neural network 300 on a batch of training examples, the embedding training system 400 processes each surface patch from each training example in the batch using the embedding neural network 300 to generate an embedding of the surface patch.

In particular, for each positive training example in a batch, the embedding training system 400A processes (in accordance with current values of the learnable parameters of the embedding neural network 300A) the binder surface patch 402A and the target surface patch 404A from the training example to generate an embedding 410A of the binder surface patch 402A and an embedding 412A of the target surface patch 404A.

For each negative training example, the embedding training system 400A processes (in accordance with current values of the learnable parameters of the embedding neural network 300A) the target surface patch 404A and the random surface patch 406A from the training example to generate an embedding 412A of the binder surface patch 402A and an embedding 414A of the random surface patch 406A.

To train degron structural classification neural network 300B, the embedding neural network receives surfaces of known substrates of the E3 ligase with the vertices corresponding to the degron labeled, for example, with a 1, and those not corresponding to a degron labeled, for example, with a 0. This labeling is referred to as the ground truth An additional set of augmented surfaces for training can be obtained by, for example, using degron identification system 200A. The neural network is trained by predicting the degron region in the input surface, with the input consisting of the features at each surface point, along with the angular and radial coordinates, and the output consisting of the prediction at each vertex. The output is compared using a loss function, for example binary cross entropy, and the neural network weights are updated using backpropagation to minimize the difference between the predicted degron surface and the known degron surface.

Optionally, before providing the surface patches from a training example to the embedding neural network 300A, the embedding training system 400A can invert some or all of the features associated with the vertices of one of the surface patches. “Inverting” a feature can refer to, e.g., scaling the feature by a negative value, e.g., the value −1. In one example, the embedding training system 400A can, for each vertex in a surface patch, invert all the features associated with the vertex except for the hydropathy feature. Inverting the features of one of the surface patches in each training example reflects that protein surface patches interact as a result of being complementary, and inverting the features of one surface patch encodes this complementarity in the data processed by the embedding neural network 300.

The embedding training system 400A determines gradients of an objective function that depends on the embeddings of the surface patches from the training examples, and uses the gradients to update the current values of the embedding neural network 300 parameters. Any appropriate gradient descent optimization procedure may be employed, e.g., RMSprop or Adam. With each training iteration, the training system 400 thus processes a batch of samples with the embedding neural network 300A according to the most recent (updated) values of the learnable parameters of network 300A. Generally, the objective function encourages the embedding neural network 300A to generate similar embeddings of interacting protein surface patches, i.e., from positive training examples. Conversely, the objective function encourages the embedding neural network 300A to generate dissimilar embeddings of non-interacting protein surface patches, i.e., from negative training examples.

In some examples, the objective function [symbol] can be given by, e.g.:

$$ℒ = {σ}_{p} + {σ}_{n} + {μ}_{p} + max ( 0 , M - {μ}_{n} ) \qquad ( 3 )$$

where μₚ is the median distance between embeddings of binder and target surface patches in the current batch, σₚ is the standard deviation of the distances between embeddings of binder and target surface patches in the current batch, μₙ is the median distance between embeddings of target and random surface patches in the current batch, σₙ is the standard deviation of the distances between embeddings of target and random surface patches in the current batch, and M is a hyper-parameter. Training of the embedding neural network 300A is deemed completed once a training completion condition is achieved, e.g., upon completing at least a defined number of training iterations, upon processing at least a defined number of training examples, upon consuming all available training examples, or upon reaching a point at which the marginal improvement in predictive power of the embedding neural network 300A between successive training iterations no longer satisfies a minimal improvement criterion.

When the embedding neural network 300A is in use by the degron identification system 200A after training, the features of surface patches are generally not inverted prior to the surface patch between processed by the embedding neural network 300A. This reflects that the degron identification system seeks to identify candidate surface patches that are similar to degron surface patches, rather than being complementary to degron surface patches.

Conversely, when the embedding neural network 300A is in use by the degron identification system 200B after training, the features of surface patches are generally inverted prior to the surface patch being processed by the embedding neural network 300A. This reflects that the degron identification system seeks to identify candidate surface patches that are complementary to E3 ligase surface patches, rather than being similar to E3 ligase surface patches.

FIG. 5 shows an example filtering system 500. The filtering system 500 is an example of a system implemented as computer programs on one or more computers in one or more locations in which the systems, components, and techniques described below are implemented.

The filtering system 500 is configured to receive a set of predicted degron surface patches 106, e.g., from the degron identification system described with reference to FIG. 2, and to identify and remove any false positives from the set of predicted degron surface patches. (A predicted degron surface patch is referred to as a false positive if it is incorrectly identified as corresponding to degron on a protein molecular surface).

The filtering system 500 includes a structural classification neural network 502 and a filtering engine 506, which are each described in more detail next.

The filtering neural network 502 is configured to process data defining a surface patch from a protein molecular surface, in accordance with values of a set of structural classification neural network parameters, to generate a filtering score 504. The filtering score 504 is a numerical value that defines a likelihood that the input surface patch includes a specified structural feature. The specified structural feature may be a structural feature that is known to be characteristic of degrons, e.g., a G-loop.

The filtering system 500 processes each predicted degron surface patch 106 using the structural classification neural network 502 to generate a corresponding filtering score 504 for the predicted degron surface patch 106.

The filtering engine 506 is configured to process the filtering scores 504 to determine whether any of the predicted degron surface patches 106 are associated with filtering scores that fail to satisfy (e.g., exceed) a threshold. If the filtering engine 506 determines that a predicted degron surface patch is associated with filtering score 504 that fails to satisfy the threshold, then the filtering engine 506 can identify the predicted degron surface patch as a false positive and remove it from the set of predicted degron surface patches 106.

The value of the threshold can be set in any appropriate way. Setting the threshold to a higher value may establish a more stringent criterion that results in more predicted degron surface patches being identified as false positives and removed from the set of predicted degron surface patches 106.

The filtering system 500 increases the accuracy and quality of the set of predicted degron surface patches 106 by removing surface patches that do not include a specified structural feature, e.g., that is known to be characteristic of degrons.

The purpose of the filtering system 500 is to further select only surface-based degrons that have high surface mimicry to a G-loop. The filtering system is then applied to all the degrons identified based on their surface. Only those surface degrons that achieve a high score (e.g., >0.9 or another specified threshold) by the filtering neural network in 502 are accepted as surface degrons, while those below this score are discarded. A classification training system 508 can train the filtering neural network 502 on a set of training data 510. The training data 510 can include a set of training examples, where each training example includes: (i) a surface patch from a protein molecular surface, and (ii) a target score indicating whether the surface patch includes a specified structural feature, e.g., a G-loop.

The classification training system 508 can train the filtering neural network 502 on the training data 510 over a sequence of training iterations.

Prior to the first training iteration, the classification training system 508 can initialize the values of the filtering neural network parameters, e.g., by randomly initializing the value of each filtering neural network parameter.

At each training iteration, the classification training system 508 can sample a batch (set) of training examples from the training data 510, and train the filtering neural network 502 on each training example in the batch.

To train the filtering neural network 502 on a training example, the classification training system 508 processes the surface patch from the training example using the filtering neural network 502 to generate a corresponding filtering score 504. The classification training system 508 determines gradients, with respect to the parameter values of the filtering neural network, of an objective function that measures an error between: (i) the filtering score, and (ii) the target score specified by the training example, e.g., by backpropagation. The classification training system 508 then updates the current parameter values of the structural classification neural network using the gradients of the objective function, e.g., by any appropriate gradient descent optimization procedure, e.g., RMSprop or Adam. The classification training system 508 thus trains the filtering neural network 502 to generate the target scores specified by the training examples.

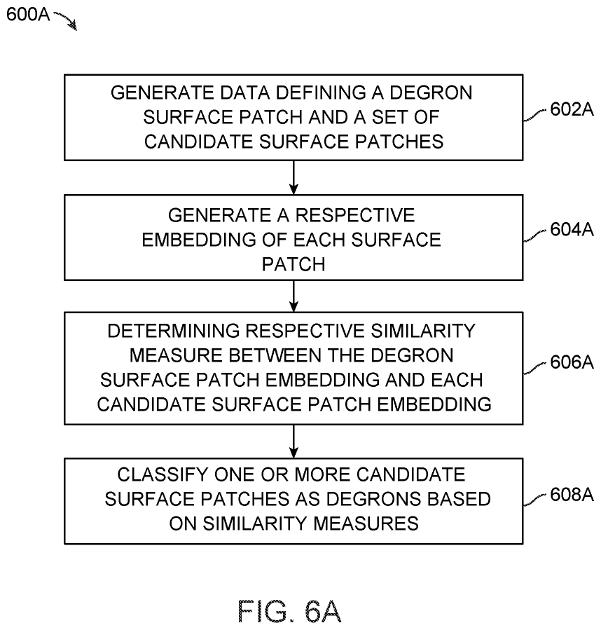

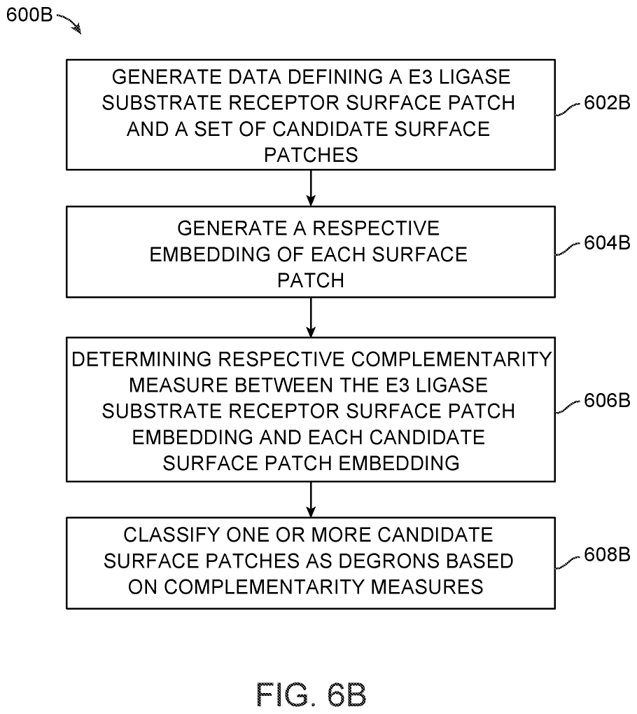

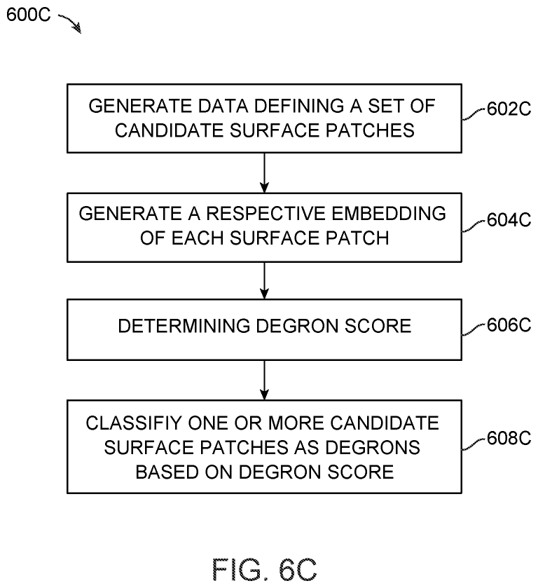

FIG. 6A is a flow diagram of an example process 600A for classifying one or more candidate protein molecular surface patches as corresponding to degrons. For convenience, the process 600A will be described as being performed by a system of one or more computers located in one or more locations. For example, a degron identification system, e.g., the degron identification system 200A of FIG. 2A, appropriately programmed in accordance with this specification, can perform the process 600A.

The system generates data defining surface patches that each represent a region on a respective protein molecular surface (602A). The surface patches include: (i) a degron surface patch that corresponds to a degron on a protein molecular surface, and (ii) a set of candidate surface patches.

The system generates a respective embedding for each of the surface patches, including, for each surface patch, processing data defining the surface patch using an embedding neural network to generate an embedding of the surface patch in an embedding space (604A).

The system determines, for each of the candidate surface patches, a respective similarity measure between: (i) the embedding of the candidate surface patch, and (ii) the embedding of the degron surface patch (606A).

The system classifies one or more of the candidate surface patches as corresponding to degrons based on the similarity measures (608A). In some implementations, the subset of candidate surface patches that are predicted to be degrons are filtered, e.g., by a degron structural classification neural network, to remove predicted degrons that are likely false positives. The system can further output an indication of unfiltered set of predicted degrons, filtered set of predicted degrons, or both. For example, the system can provide the filtered or unfiltered set of predicted degrons for presentation to a user (e.g., on an electronic display screen), transmit data identifying predicted degrons to specified accounts or devices associated with one or more users, and may store data indicating the filtered or unfiltered set of predicted degrons on computer-readable memory or storage devices.

FIG. 6B is a flow diagram of an example process 600B for classifying one or more candidate protein molecular surface patches as corresponding to degrons. For convenience, the process 600B will be described as being performed by a system of one or more computers located in one or more locations. For example, a degron identification system, e.g., the degron identification system 200B of FIG. 2B, appropriately programmed in accordance with this specification, can perform the process 600B. The system generates data defining surface patches that each represent a region on a respective protein molecular surface (602B). The surface patches include: (i) an E3 ligase surface patch that corresponds to a molecular surface or neosurface of an E3 ligase substrate receptor protein, and (ii) a set of candidate surface patches.

The system generates a respective embedding for each of the surface patches, including, for each surface patch, processing data defining the surface patch using an embedding neural network to generate an embedding of the surface patch in an embedding space (604B).

The system determines, for each of the candidate surface patches, a respective complementarity measure between: (i) the embedding of the candidate surface patch, and (ii) the embedding of the E3 ligase surface patch (606B).

The system classifies one or more of the candidate surface patches as corresponding to degrons based on the similarity measures (608A). In some implementations, the subset of candidate surface patches that are predicted to be degrons are filtered, e.g., by a degron structural classification neural network, to remove predicted degrons that are likely false positives. The system can further output an indication of unfiltered set of predicted degrons, filtered set of predicted degrons, or both. For example, the system can provide the filtered or unfiltered set of predicted degrons for presentation to a user (e.g., on an electronic display screen), transmit data identifying predicted degrons to specified accounts or devices associated with one or more users, and may store data indicating the filtered or unfiltered set of predicted degrons on computer-readable memory or storage devices.

FIG. 6C is a flow diagram of an example process 600C for classifying one or more candidate protein molecular surface patches as corresponding to degrons. For convenience, the process 600C will be described as being performed by a system of one or more computers located in one or more locations. For example, a degron identification system, e.g., the degron identification system 200C of FIG. 2C, appropriately programmed in accordance with this specification, can perform the process of 600C. The system generates data defining surface patches that each represent a region on a respective protein molecular surface (602C). The surface patches include a set of candidate surface patches.

The system generates a respective embedding for each of the candidate surface patches, including, for each surface patch, processing data defining the surface patch using an embedding neural network to generate an embedding of the surface patch in an embedding space (604C).

The system determines, for each of the candidate surface patches, a degron score (606C).

The system classifies one or more of the candidate surface patches as corresponding to degrons based on the similarity measures (608C). In some implementations, the subset of candidate surface patches that are predicted to be degrons are filtered, e.g., by a degron structural classification neural network, to remove predicted degrons that are likely false positives. The system can further output an indication of unfiltered set of predicted degrons, filtered set of predicted degrons, or both. For example, the system can provide the filtered or unfiltered set of predicted degrons for presentation to a user (e.g., on an electronic display screen), transmit data identifying predicted degrons to specified accounts or devices associated with one or more users, and may store data indicating the filtered or unfiltered set of predicted degrons on computer-readable memory or storage devices. This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

### EXAMPLES

The following examples are included for illustrative purposes only and are not intended to limit the scope of the invention.

### Example 1: Identification of Neosubstrates

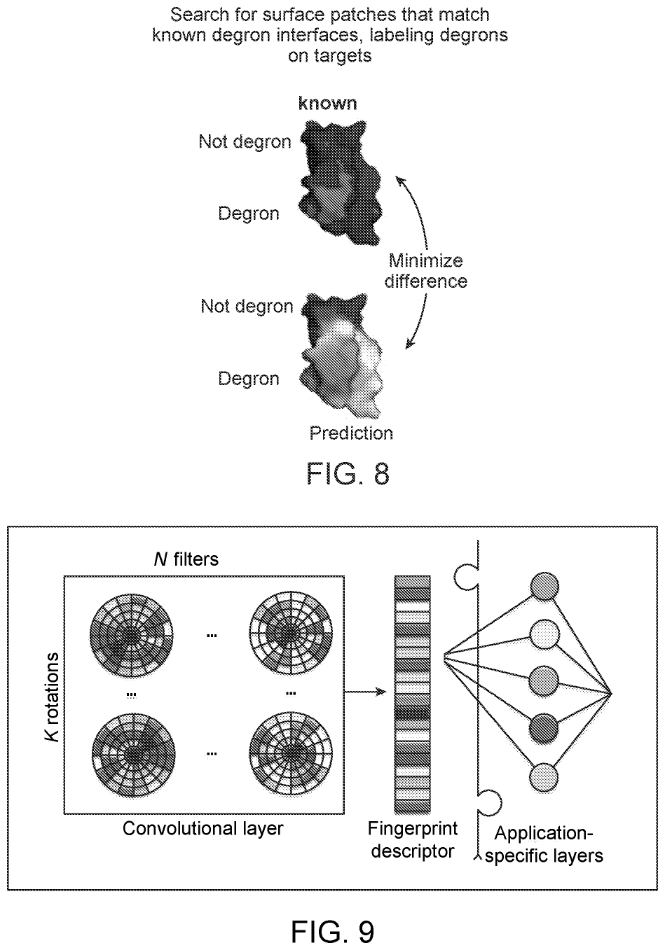

FIG. 8 shows an example of training a degron identification machine learning agent using surface patches.

FIG. 9 shows a conceptual example of using an ultra-fast fingerprint search for similar surfaces, finding surface that mimic known degron surfaces.

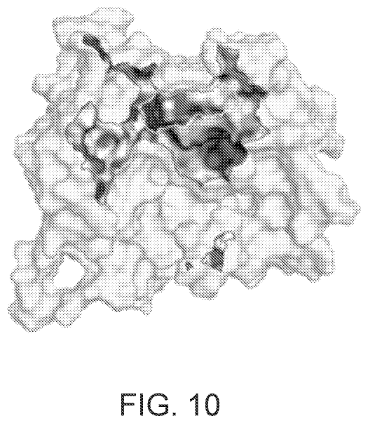

FIG. 10 depicts a E3:MGD neosurface for an ultra-fast fingerprint search for complementary surfaces, such as for E3 ligase-neosubstrate matchmaking.

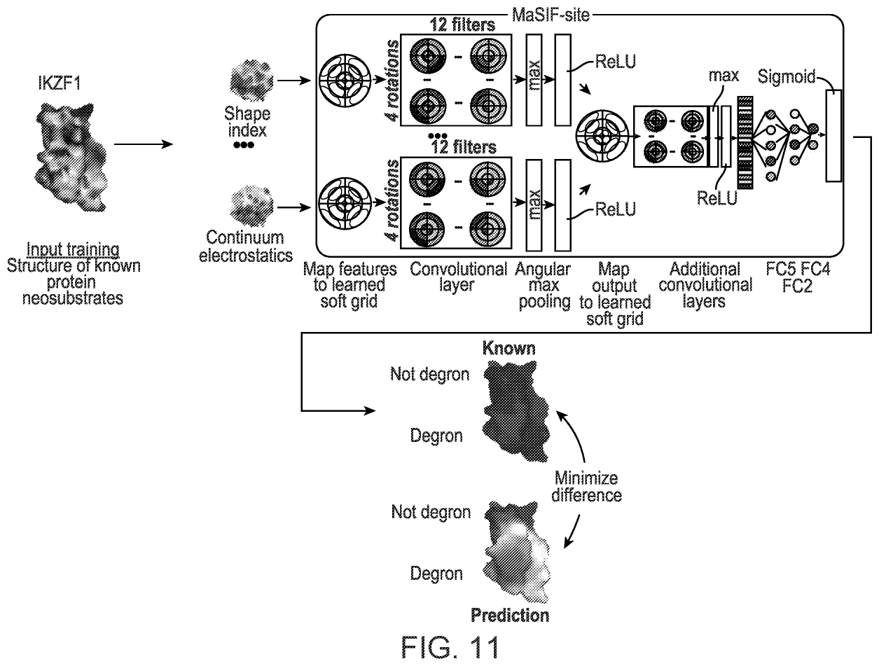

FIG. 11 depicts an example of a method for learning CRBN degron features from known degron surfaces. The algorithm classifies protein surfaces for the presence of degrons. The algorithm creates a feature-rich surface characterization and uses 3 layers of geodesic convolution with deep vertexes to classify input surfaces.

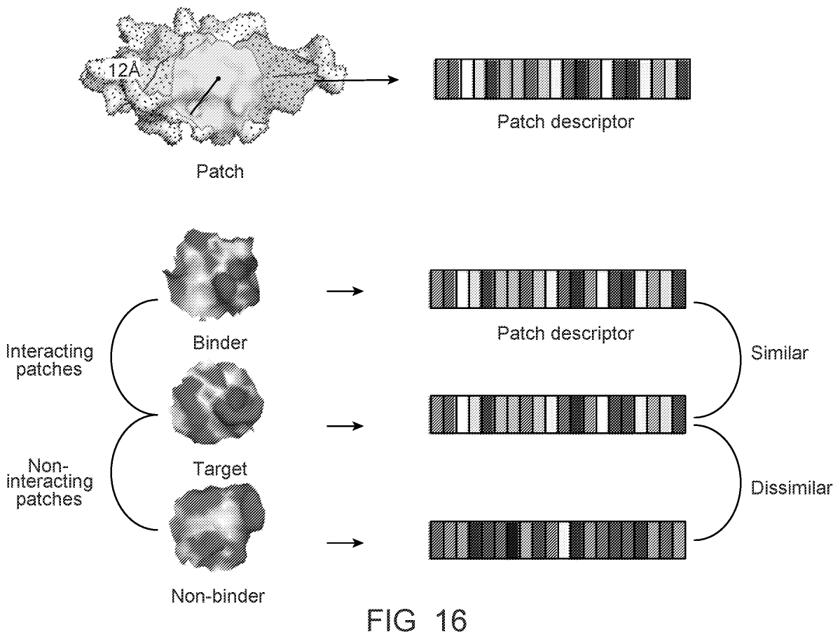

FIG. 16 shows an example of encoding protein surfaces as fingerprints, which enables ultra-fast, proteome-wide searching for similar & complementary fingerprints of degrons and E3 ligases respectively.

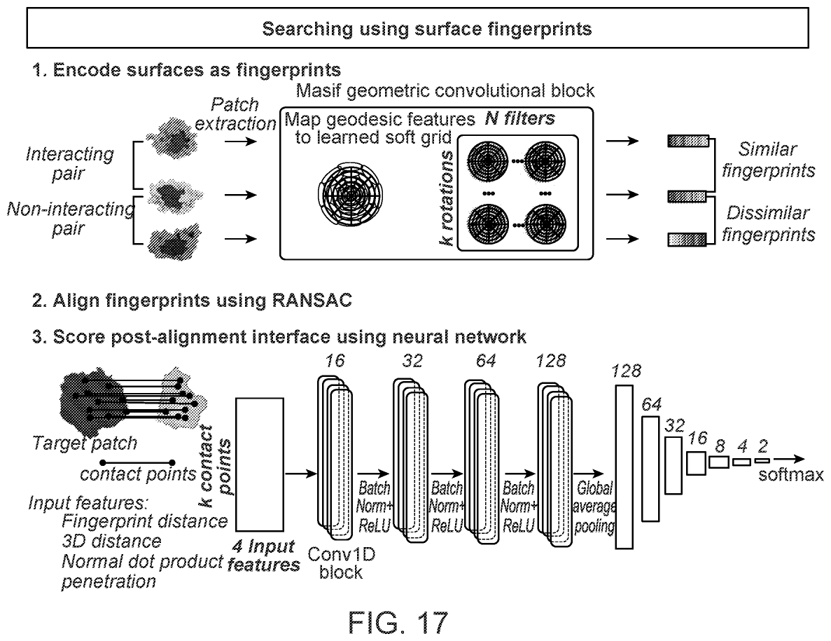

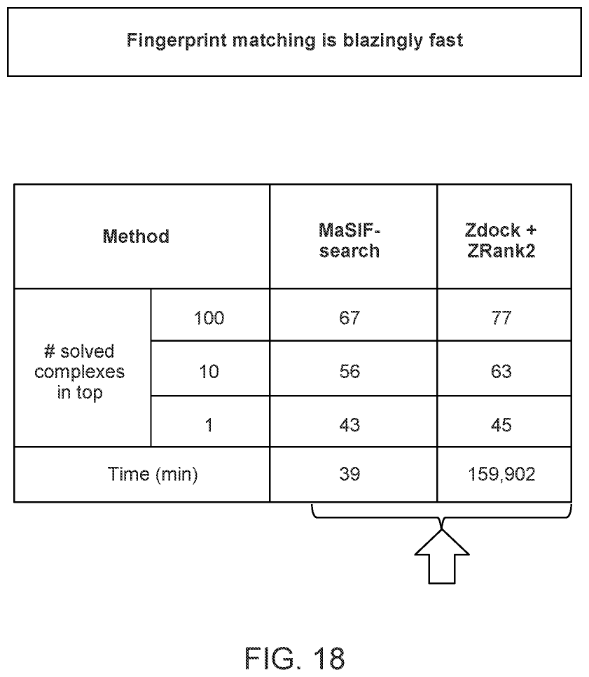

FIG. 17 shows an example of a full pipeline to search for complementary or similar degrons.

FIG. 18 shows that fingerprint encoding enables ultra-fast searching of, for example, proteome-wide queries of either complementary or similar surfaces of degrons and E3 ligases respectively.

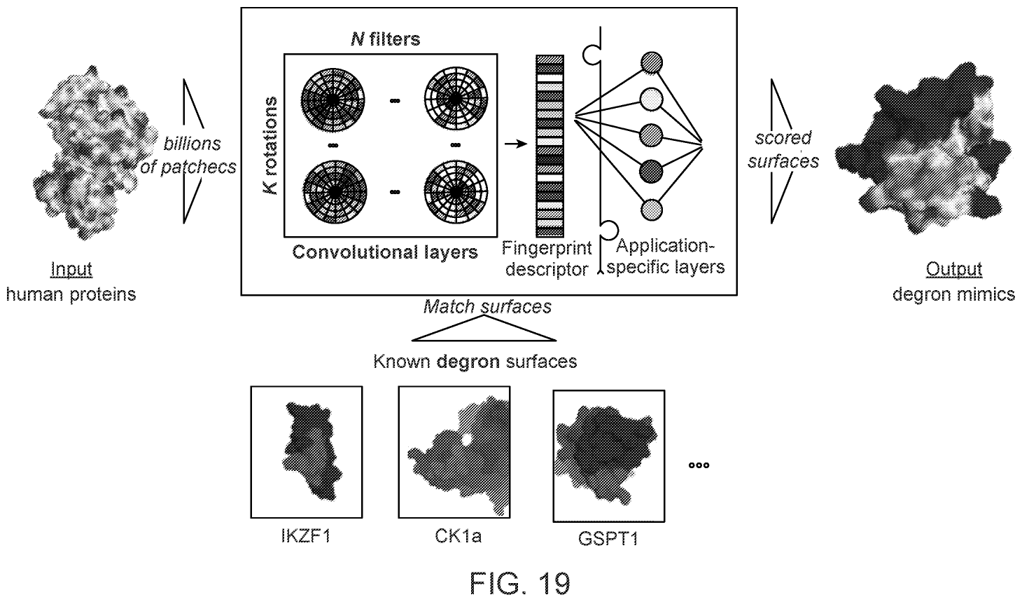

FIG. 19 shows an example of proteome-wide fast matching of degron surface mimics by matching of surface fingerprints (and not, e.g., G-loops per se), according to method 600C.

### Example 2: Identification and Validation of Neosubstrates

Putative neosubstrates of CRBN were identified as depicted in FIG. 6C and experimentally validated.

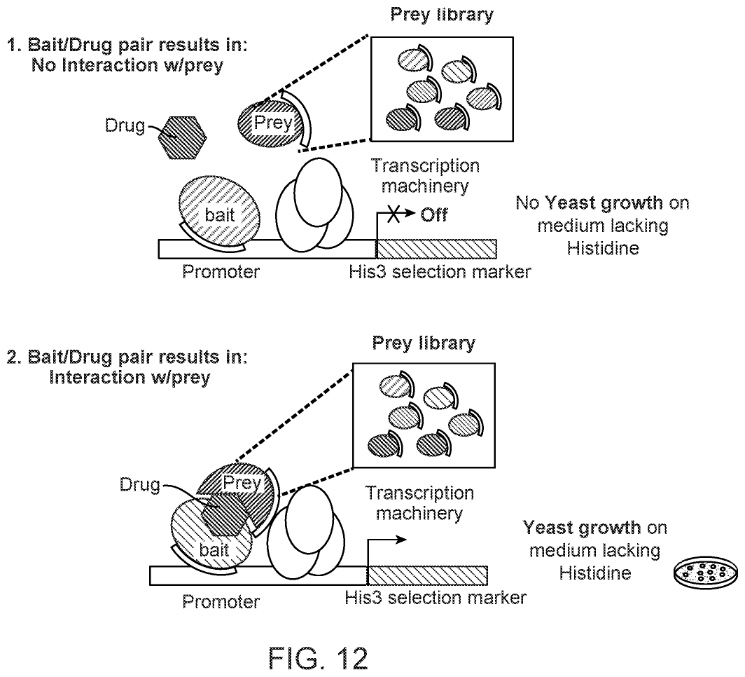

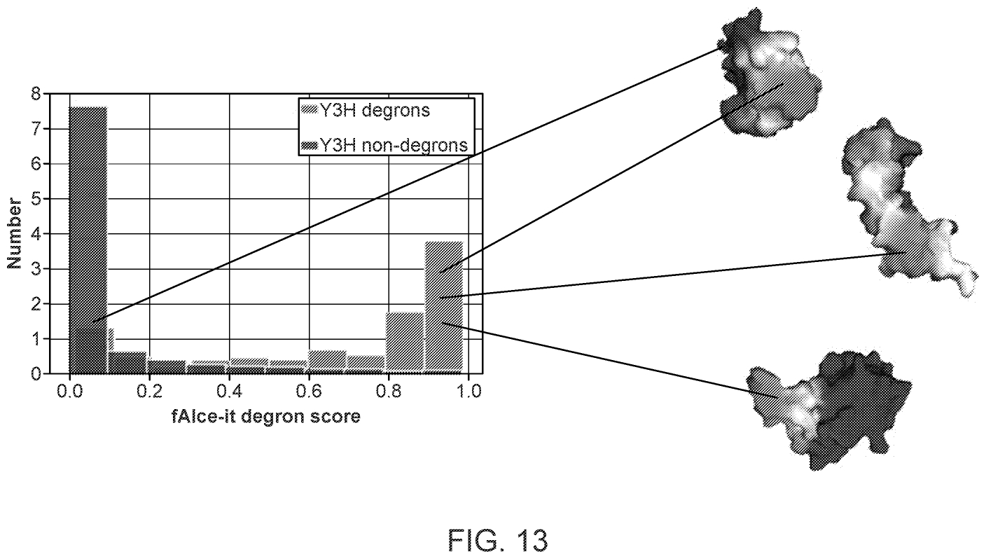

Yeast three hybrid experiments were carried out to identify molecular glue induced interactions between CRBN and cDNA library-derived targets, as depicted in FIG. 12, which allowed mapping degrons to individual protein domains. The experiments identified 8 novel G-loops from 5 distinct domain classes, which agreed with predictions generated using the degron identification system, as shown in FIG. 13.

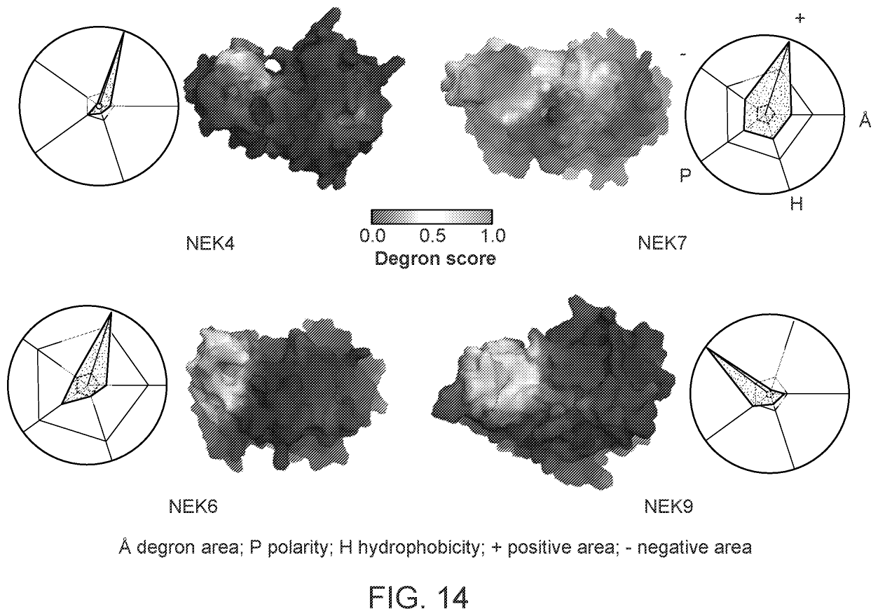

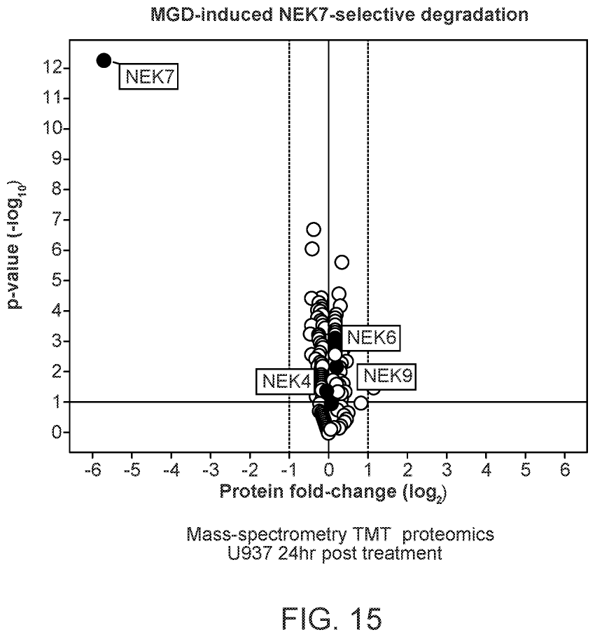

As shown in FIG. 14, a unique G-loop surface was identified for NEK7, which allows selective MGD degradation, as shown in FIG. 15.

### Example 3: Identification and Validation of Neosubstrates

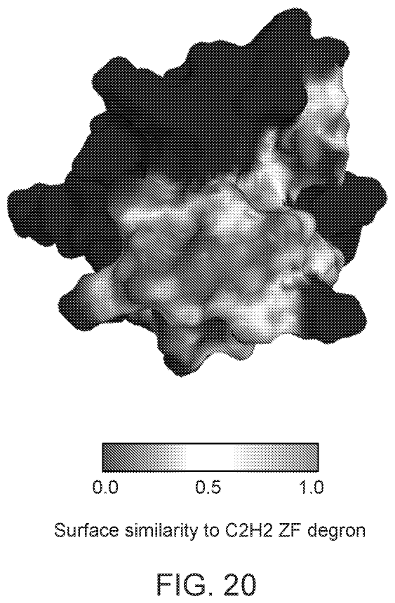

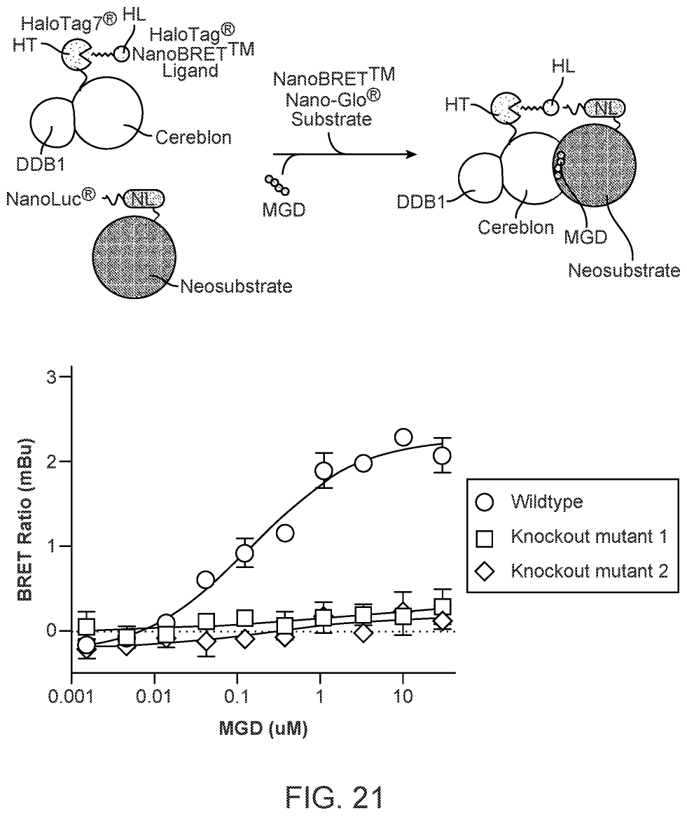

Putative neosubstrates of CRBN were identified as depicted in FIG. 6A and experimentally validated. As shown in FIG. 20, a novel non-hairpin, non-canonical degron in an established oncology target (with surface similarity to C2H2 ZF degron), was identified by proteome-wide fast matching of degron surface mimics (i.e., surface fingerprint matching as opposed to G-loop identification). As shown in FIG. 21, NanoBRET confirmed the prediction and binding mode.

### Example 4: Identification and Validation of Neosubstrates

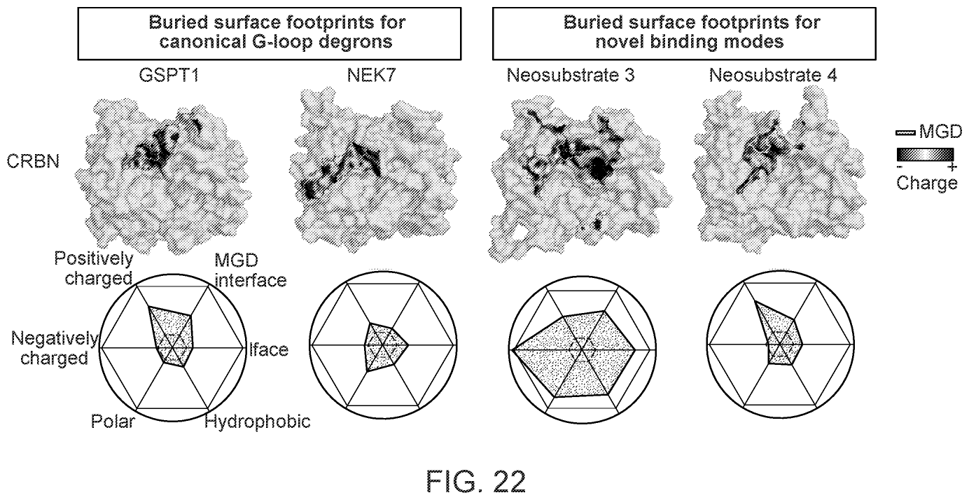

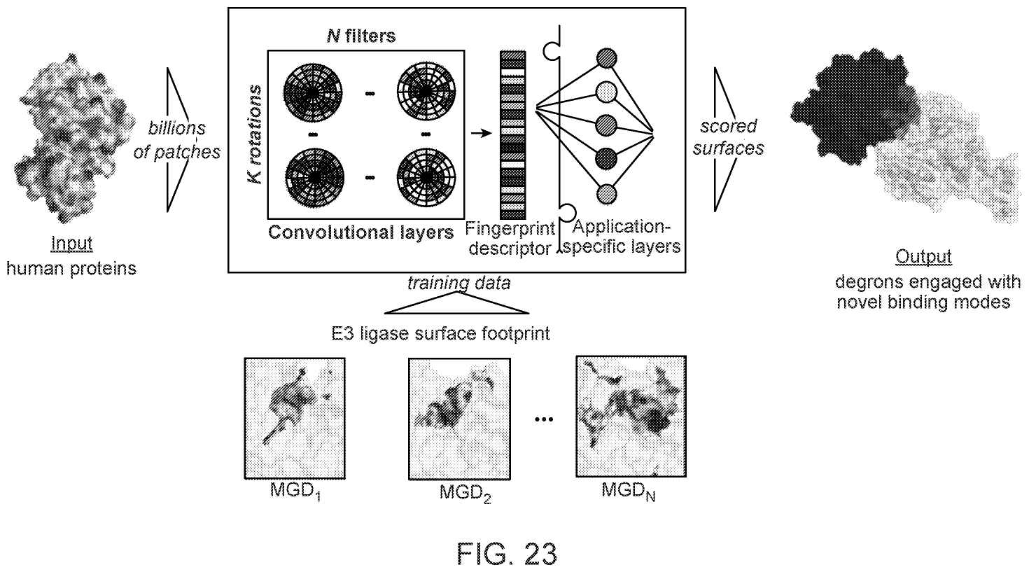

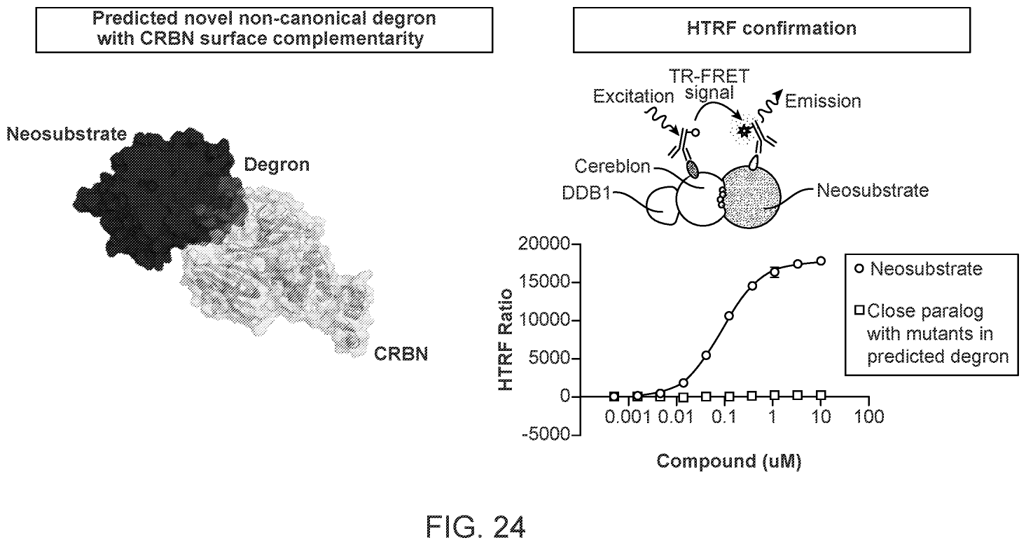

Putative neosubstrates of CRBN were identified as depicted in FIG. 6B and experimentally validated. The CRBN neosurface was used to find novel substrates (e.g., as depicted in FIG. 22 and FIG. 23), and validated in an HTRF assay (e.g., as depicted in FIG. 24).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method performed by one or more computers, the method comprising:
obtaining data defining a plurality of surface patches that each represent a region on a respective protein molecular surface, wherein the plurality of surface patches comprise: (i) a degron surface patch that corresponds to a degron on a protein molecular surface, and (ii) a plurality of candidate surface patches;
generating a respective embedding for each of the plurality of surface patches, comprising, for each surface patch:
processing data defining the surface patch using an embedding neural network to generate an embedding of the surface patch in an embedding space;
determining, for each of the plurality of candidate surface patches, a respective similarity measure between: (i) the embedding of the candidate surface patch, and (ii) the embedding of the degron surface patch; and
classifying one or more of the candidate surface patches as corresponding to degrons based on the similarity measures.

2. The method of claim 1, wherein classifying one or more of the candidate surface patches as corresponding to degrons comprises:
classifying each candidate surface patch for which the similarity measure between: (i) the embedding of the candidate surface patch, and (ii) the embedding of the degron surface patch, satisfies a threshold as representing as corresponding to a degron.

3. The method of claim 1, further comprising:
generating, for each of the candidate surface patches that have been classified as corresponding to a degron, a respective filtering score defining a probability that the protein molecular surface region represented by the candidate surface patch includes a specified structural feature; and
filtering the candidate surface patches that have been classified as corresponding to degrons to remove candidate surface patches for which the filtering score fails to satisfy a threshold.

4. The method of claim 3, wherein the specified structural feature comprises a G-loop.

5. The method of claim 3, wherein for each candidate surface patch that is classified as corresponding to a degron, generating the filtering score for the candidate surface patch comprises:
processing data defining the candidate surface patch using a structural classification neural network to generate the filtering score,
wherein the structural classification neural network has been trained to classify whether an input surface patch represents a protein molecular surface region that includes the specified structural feature.

6. The method of claim 1, wherein for each of the plurality of surface patches, obtaining data defining the surface patch comprises:
generating a discrete representation of a protein molecular surface as a polygon mesh comprising a set of vertices;
generating a respective set of features corresponding to each vertex in the polygon mesh; and
identifying the surface patch as representing a region of the polygon mesh within a predefined geodesic distance of a center point on the polygon mesh, wherein the data defining the surface patch includes, for each vertex included in the region of the polygon mesh: (i) coordinates of the vertex, and (ii) the set of features corresponding to the vertex.

7. The method of claim 6, wherein for each vertex in the polygon mesh, generating the respective set of features corresponding to the vertex comprises:
generating one or more chemical features that characterize biochemical properties of the protein molecular surface at the vertex.

8. The method of claim 7, wherein the chemical features include one or more of: a hydropathy index feature, a continuum electrostatics feature, or a hydrogen bonding potential feature.

9. The method of claim 6, wherein for each vertex in the polygon mesh, generating the respective set of features corresponding to the vertex comprises:
generating one or more geometric features that characterize a shape of the protein molecular surface at the vertex.

10. The method of claim 9, wherein the geometric features comprise a shape index feature, a distance-dependent curvature feature, or both.

11. The method of claim 1, wherein the embedding neural network has been trained by a plurality of operations comprising:
processing data defining a first surface patch representing a first protein molecular surface region using the embedding neural network to generate an embedding of the first surface patch;
processing data defining a second surface patch representing a second protein molecular surface region using the embedding neural network to generate an embedding for the second surface patch; and
adjusting values of a plurality of neural network parameters of the embedding neural network using gradients of an objective function that measures a distance between the embedding of the first surface patch and the embedding of the second surface patch.

12. The method of claim 11, wherein the first protein molecular surface region interacts with the second protein molecular surface region, and the objective function encourages a higher similarity between the embedding of the first surface patch and the embedding of the second surface patch.

13. The method of claim 11, wherein the first protein molecular surface region does not interact with the second protein molecular surface region, and the objective function encourages a lower similarity between the embedding of the first surface patch and the embedding of the second surface patch.

14. The method of claim 11, further comprising, prior to processing the data defining the second surface patch using the embedding neural network:
inverting, for each vertex included in the second surface patch, at least some features in a set of features associated with the vertex.

15. The method of claim 14, wherein inverting a feature comprises scaling the feature value by a negative value.

16. A system comprising:
one or more computers; and
one or more storage devices communicatively coupled to the one or more computers, wherein the one or more storage devices store instructions that, when executed by the one or more computers, cause the one or more computers to perform operations comprising:
obtaining data defining a plurality of surface patches that each represent a region on a respective protein molecular surface, wherein the plurality of surface patches comprise: (i) a degron patch that corresponds to a degron on a protein molecular surface, and (ii) a plurality of candidate surface patches;
generating a respective embedding for each of the plurality of surface patches, comprising, for each surface patch:
processing data defining the surface patch using an embedding neural network to generate an embedding of the surface patch in an embedding space;
determining, for each of the plurality of candidate surface patches, a respective similarity measure between: (i) the embedding of the candidate surface patch, and (ii) the embedding of the degron surface patch; and
classifying one or more of the candidate surface patches as corresponding to degrons based on the similarity measures.

17. One or more non-transitory computer storage media storing instructions that when executed by one or more computers cause the one or more computers to perform operations comprising:
obtaining data defining a plurality of surface patches that each represent a region on a respective protein molecular surface, wherein the plurality of surface patches comprise: (i) a degron patch that corresponds to a degron on a protein molecular surface, and (ii) a plurality of candidate surface patches;
generating a respective embedding for each of the plurality of surface patches, comprising, for each surface patch:
processing data defining the surface patch using an embedding neural network to generate an embedding of the surface patch in an embedding space;
determining, for each of the plurality of candidate surface patches, a respective similarity measure between: (i) the embedding of the candidate surface patch, and (ii) the embedding of the degron surface patch; and
classifying one or more of the candidate surface patches as corresponding to degrons based on the similarity measures.

18. A method performed by one or more computers, the method comprising:
obtaining data defining a plurality of surface patches that each represent a region on a respective protein molecular surface, wherein the plurality of surface patches comprise: (i) a E3 ligase surface patch that corresponds to an E3 ligase surface or neosurface, and (ii) a plurality of candidate surface patches;
generating a respective embedding for each of the plurality of surface patches, comprising, for each surface patch:
processing data defining the surface patch using an embedding neural network to generate an embedding of the surface patch in an embedding space;
determining, for each of the plurality of candidate surface patches, a respective complementarity measure between: (i) the embedding of the candidate surface patch, and (ii) the embedding of the E3 ligase surface patch; and
classifying one or more of the candidate surface patches as corresponding to degrons based on the complementarity measures.

19. The method of claim 18, wherein classifying one or more of the candidate surface patches as corresponding to degrons comprises:
classifying each candidate surface patch for which the complementarity measure between: (i) the embedding of the candidate surface patch, and (ii) the embedding of the E3 ligase surface patch, satisfies a threshold as representing as corresponding to a degron.

20. The method of claim 18, further comprising:
generating, for each of the candidate surface patches that have been classified as corresponding to a degron, a respective filtering score defining a probability that the protein molecular surface region represented by the candidate surface patch includes a specified structural feature; and
filtering the candidate surface patches that have been classified as corresponding to degrons to remove candidate surface patches for which the filtering score fails to satisfy a threshold.

21. The method of claim 20, wherein the specified structural feature comprises a G-loop.

22. The method of claim 20, wherein for each candidate surface patch that is classified as corresponding to a degron, generating the filtering score for the candidate surface patch comprises:
processing data defining the candidate surface patch using a structural classification neural network to generate the filtering score,
wherein the structural classification neural network has been trained to classify whether an input surface patch represents a protein molecular surface region that includes the specified structural feature.

23. The method of claim 18, wherein for each of the plurality of surface patches, obtaining data defining the surface patch comprises:
generating a discrete representation of a protein molecular surface as a polygon mesh comprising a set of vertices;
generating a respective set of features corresponding to each vertex in the polygon mesh; and
identifying the surface patch as representing a region of the polygon mesh within a predefined geodesic distance of a center point on the polygon mesh, wherein the data defining the surface patch includes, for each vertex included in the region of the polygon mesh: (i) coordinates of the vertex, and (ii) the set of features corresponding to the vertex.

24. The method of claim 23, wherein for each vertex in the polygon mesh, generating the respective set of features corresponding to the vertex comprises:
generating one or more chemical features that characterize biochemical properties of the protein molecular surface at the vertex.

25. The method of claim 24, wherein the chemical features include one or more of: a hydropathy index feature, a continuum electrostatics feature, or a hydrogen bonding potential feature.

26. The method of claim 23, wherein for each vertex in the polygon mesh, generating the respective set of features corresponding to the vertex comprises:
generating one or more geometric features that characterize a shape of the protein molecular surface at the vertex.

27. The method of claim 26, wherein the geometric features comprise a shape index feature, a distance-dependent curvature feature, or both.

28. The method of claim 18, wherein the embedding neural network has been trained by a plurality of operations comprising:
processing data defining a first surface patch representing a first protein molecular surface region using the embedding neural network to generate an embedding of the first surface patch;
processing data defining a second surface patch representing a second protein molecular surface region using the embedding neural network to generate an embedding for the second surface patch; and
adjusting values of a plurality of neural network parameters of the embedding neural network using gradients of an objective function that measures a distance between the embedding of the first surface patch and the embedding of the second surface patch.

29. The method of claim 28, wherein the first protein molecular surface region interacts with the second protein molecular surface region, and the objective function encourages a higher complementarity between the embedding of the first surface patch and the embedding of the second surface patch.

30. The method of claim 28, wherein the first protein molecular surface region does not interact with the second protein molecular surface region, and the objective function encourages a lower complementarity between the embedding of the first surface patch and the embedding of the second surface patch.

31. A system comprising:
one or more computers; and
one or more storage devices communicatively coupled to the one or more computers, wherein the one or more storage devices store instructions that, when executed by the one or more computers, cause the one or more computers to perform operations comprising:
obtaining data defining a plurality of surface patches that each represent a region on a respective protein molecular surface, wherein the plurality of surface patches comprise: (i) a E3 ligase patch that corresponds to an E3 ligase surface or neosurface, and (ii) a plurality of candidate surface patches;
generating a respective embedding for each of the plurality of surface patches, comprising, for each surface patch:
processing data defining the surface patch using an embedding neural network to generate an embedding of the surface patch in an embedding space;
determining, for each of the plurality of candidate surface patches, a respective similarity measure between: (i) the embedding of the candidate surface patch, and (ii) the embedding of the E3 ligase surface patch; and
classifying one or more of the candidate surface patches as corresponding to degrons based on the similarity measures.

32. One or more non-transitory computer storage media storing instructions that when executed by one or more computers cause the one or more computers to perform operations comprising:
obtaining data defining a plurality of surface patches that each represent a region on a respective protein molecular surface, wherein the plurality of surface patches comprise: (i) a E3 ligase patch that corresponds to an E3 ligase surface or neosurface, and (ii) a plurality of candidate surface patches;
generating a respective embedding for each of the plurality of surface patches, comprising, for each surface patch:
processing data defining the surface patch using an embedding neural network to generate an embedding of the surface patch in an embedding space;
determining, for each of the plurality of candidate surface patches, a respective similarity measure between: (i) the embedding of the candidate surface patch, and (ii) the embedding of the E3 ligase surface patch; and
classifying one or more of the candidate surface patches as corresponding to degrons based on the similarity measures.

33. A method performed by one or more computers, the method comprising:
obtaining data defining a plurality of surface patches that represent a region on a respective protein molecular surface, wherein the plurality of surface patches comprising a plurality of candidate surface patches;
generating a respective embedding for each of the plurality of surface patches, comprising, for each surface patch:
processing data defining the surface patch using an embedding neural network to generate an embedding of the surface patch in an embedding space;
determining, for each of the plurality of candidate surface patches, a degron score; and
classifying one or more of the candidate surface patches as corresponding to degrons based on the degron score.

34. The method of claim 33, wherein classifying one or more of the candidate surface patches as corresponding to degrons comprises:
classifying each candidate surface patch for which the degron score satisfies a threshold as representing as corresponding to a degron.

35. The method of claim 33, further comprising:
generating, for each of the candidate surface patches that have been classified as corresponding to a degron, a respective filtering score defining a probability that the protein molecular surface region represented by the candidate surface patch includes a specified structural feature; and
filtering the candidate surface patches that have been classified as corresponding to degrons to remove candidate surface patches for which the filtering score fails to satisfy a threshold.

36. The method of claim 35, wherein the specified structural feature comprises a G-loop.

37. The method of claim 35, wherein for each candidate surface patch that is classified as corresponding to a degron, generating the filtering score for the candidate surface patch comprises:
processing data defining the candidate surface patch using a structural classification neural network to generate the filtering score,
wherein the structural classification neural network has been trained to classify whether an input surface patch represents a protein molecular surface region that includes the specified structural feature.

38. The method of claim 33, wherein for each of the plurality of surface patches, obtaining data defining the surface patch comprises:
generating a discrete representation of a protein molecular surface as a polygon mesh comprising a set of vertices;
generating a respective set of features corresponding to each vertex in the polygon mesh; and
identifying the surface patch as representing a region of the polygon mesh within a predefined geodesic distance of a center point on the polygon mesh, wherein the data defining the surface patch includes, for each vertex included in the region of the polygon mesh: (i) coordinates of the vertex, and (ii) the set of features corresponding to the vertex.

39. The method of claim 38, wherein for each vertex in the polygon mesh, generating the respective set of features corresponding to the vertex comprises:
generating one or more chemical features that characterize biochemical properties of the protein molecular surface at the vertex.

40. The method of claim 39, wherein the chemical features include one or more of: a hydropathy index feature, a continuum electrostatics feature, or a hydrogen bonding potential feature.

41. The method of claim 38, wherein for each vertex in the polygon mesh, generating the respective set of features corresponding to the vertex comprises:
generating one or more geometric features that characterize a shape of the protein molecular surface at the vertex.

42. The method of claim 41, wherein the geometric features comprise a shape index feature, a distance-dependent curvature feature, or both.

43. The method of claim 33, wherein the embedding neural network has been trained by a plurality of operations comprising:
processing data defining a first surface patch representing a first protein molecular surface region using the embedding neural network to generate an embedding of the first surface patch;
processing data defining a second surface patch representing a second protein molecular surface region using the embedding neural network to generate an embedding for the second surface patch; and
adjusting values of a plurality of neural network parameters of the embedding neural network using gradients of an objective function that measures a distance between the embedding of the first surface patch and the embedding of the second surface patch.

44. The method of claim 43, wherein the first protein molecular surface region interacts with the second protein molecular surface region, and the objective function encourages a higher similarity between the embedding of the first surface patch and the embedding of the second surface patch.

45. The method of claim 43, wherein the first protein molecular surface region does not interact with the second protein molecular surface region, and the objective function encourages a lower similarity between the embedding of the first surface patch and the embedding of the second surface patch.

46. A system comprising:
one or more computers; and
one or more storage devices communicatively coupled to the one or more computers, wherein the one or more storage devices store instructions that, when executed by the one or more computers, cause the one or more computers to perform operations comprising:
obtaining data defining a plurality of surface patches comprising a plurality of candidate surface patches;
generating a respective embedding for each of the plurality of surface patches, comprising, for each surface patch:
processing data defining the surface patch using an embedding neural network to generate an embedding of the surface patch in an embedding space;
determining, for each of the plurality of candidate surface patches, a degron score; and
classifying one or more of the candidate surface patches as corresponding to degrons based on the similarity measures.

47. One or more non-transitory computer storage media storing instructions that when executed by one or more computers cause the one or more computers to perform operations comprising:
obtaining data defining a plurality of surface patches comprising a plurality of candidate surface patches;
generating a respective embedding for each of the plurality of surface patches, comprising, for each surface patch:
processing data defining the surface patch using an embedding neural network to generate an embedding of the surface patch in an embedding space;
determining, for each of the plurality of candidate surface patches, a degron score; and
classifying one or more of the candidate surface patches as corresponding to degrons based on the similarity measures.
